(19) United States
(12) Patent Application Publication
(10) Pub. No.: US 2026/0043086 A1
(43) Pub. Date: Feb. 12, 2026

(54) PREDICTIVE BIOMARKER IN AVASTIN IN COLON CANCER

(71) Applicant: H. LEE MOFFITT CANCER CENTER AND RESEARCH INSTITUTE, INC., Tampa, FL (US)

(72) Inventors: Dung-Tsa CHEN, Tampa, FL (US); Mokenge MALAFA, Tampa, FL (US)

(21) Appl. No.: 19/101,289

(22) PCT Filed: Aug. 7, 2023
(86) PCT No.: PCT/US2023/029660

§ 371 Date: Feb. 5, 2025

Related U.S. Application Data
(60) Provisional application No. 63/395,801, filed on Aug. 6, 2022.

(51) Int. Cl. C12Q 1/6886; A61K 39/00; A61K 39/395

(52) U.S. Cl. CPC C12Q 1/6886; A61K 39/39558; A61K 2039/505; C12Q 2600/106; C12Q 2600/158

(57) ABSTRACT

Disclosed are kits and methods for detecting whether a patient with metastatic colorectal cancer should receive AVASTIN® (bevacizumab) in addition to chemotherapy.

This application claims the benefit of U.S. Provisional Application No. 63/395,801, filed on Aug. 6, 2022, which is incorporated herein by reference in its entirety.

### I. BACKGROUND

Colorectal cancer (CRC) accounts for 8% (n=149,500) of all new cancer cases and is the second most common cause of cancer-related death (9%; n=52,980) in the United States. Unfortunately, 22% of patients diagnosed with colon cancer have metastatic CRC (mCRC) with poor clinical outcome of a 15% 5-year survival rate, highlighting significant need of effective treatment strategy to improve prognosis. Systemic chemotherapy is the primary treatment for mCRC. The first-line conventional chemotherapy for mCRC includes fluoropyrimidine monotherapy (e.g., fluorouracil and capecitabine) and combination with a fluoropyrimidine (e.g., FOLFOX (intravenous fluorouracil, leucovorin, and oxaliplatin) and FOLFIRI (fluorouracil, leucovorin and irinotecan)). The resulting median OS ranges 17-23 months. Meanwhile, targeted therapy has been considered as an add-in option with conventional chemotherapy for first-line treatment of mCRC. One extensively characterized agent is Bevacizumab (AVASTIN®), a monoclonal antibody targeting the vascular endothelial growth factor (VEGF). Various clinical trials demonstrated its efficacy in improving progression-free survival (PFS), but not in OS with a modest response rate of 10-15%. What is needed are new ways to more effectively predict AVASTIN® treatment effect.

### II. SUMMARY

Disclosed are methods and kits related to the detection of subjects who should and should not receive administration of bevacizumab (AVASTIN®) for the treatment of cancer.

In one aspect, disclosed herein are kits used for of the treatment of a cancer (such as, for example colorectal cancer, including, but not limited to metastatic colorectal cancers (mCRC) such as, for example, stage 4 mCRC) comprising one or more probe sets comprising merck2_AA101946_x_at, merck2_AA490328_at, merck2_AF129457_at, merck2_AF289590_at, merck2_AI091276_at, merck2_AI223270_at, merck2_AI962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at, merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_032 up_at, merck2_VZV_OKA_047_up_at, merck2_XM_938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655_s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_BI045425_at, merck_BI757398_at, merck_BM676784_at, merck_BM805663_at, merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431_s_at, merck_NM_001494_at, merck_NM_001883_at, merck_NM_001993_at, merck_NM_002501_s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41_s_at, merck_VZV_OKA_ORF9_s_at, merck_X85629_at, merck_X98206_at, merck_XM_063314_at, merck_XM_210303_at, merck_XM_373233_x_at, merck_XM_378090_at, merck_XM_926011_at, merck_XM_928173_s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, merck_hCT1846664_1_at, merck_hCT2307348_at, or merck_hsa_mir_153_1_x_at

Also disclosed herein are kits of any preceding aspect, wherein the one or more probe sets bind to one or more genes comprising TIMM8A, PRDM15, LOC100130744, LANCL2, L0C101929529, NPHP3 NPHP3-ACAD11, MYO5B, ZSCAN30, ZNF462, CDON. ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 L0C101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1. In one aspect, the probes bind to at least 1, 2, 3, 4, 5, 6, 7, 8, 9, 10, 11, 12, 13, 14, 15, 16, 17, 18, 19, 20, 21, or 22 of the genes selected from the group consisting of AGR2, BEST4, CRHR2, CRK, CYP24A1, DVL2, ELFN1-AS1, F3, FIP1L1, FOXP1, ICAM2, JAK1, MAP3K14, MBD2, MIB1, MSN, NKX2-5, NODAL, THUMPD3-AS1, TIMELESS, TJP2, and/or USP46. In one aspect, the probes bind to at least 1, 2, 3, 4, 5, 6, 7, 8, 9, 10, 11, 12, 13, or 14 of the genes selected from the group consisting of BFSP2-AS1, ERICH1, HERC4, HIST2H2BF, IWS1, LINC01405, MCHR1, MTMR6, SLC30A2, SLITRK1, SRP68, SRRM4, TFCP2L1, and/or ZNF462. In one aspect, the probes bind to at least 1, 2, 3, 4, 5, 6, 7, or 8 of the genes selected from the group consisting of ERICH1, HERC4, HIST2H2BF, IWS1, MTMR6, SRP68, TFCP2L1, ZNF462 (such as, for example, HERC4, HIST2H2BF, IWS1, SRP68, and/or ZNF462). In one aspect, the probes bind to at least 1, 2, 3, 4, 5, 6, or 7 of the genes selected from the group consisting of HERC4, HIST2H2BF, IWS1, MCHR1, SLC30A2, SRP68, ZNF462. It is understood and herein contemplated that low expression of some genes can be associated with an increase in death (i.e., low survivability and thus high expression is good) including, but not limited to CLPTM1, GTPBP1, IWS1, MAP3K14, MBD2, PIH1D1, SLC25A27, TJP2.

In one aspect, disclosed herein are methods of treating, inhibiting, reducing, decreasing, ameliorating, and/or preventing a cancer and/or metastasis (such as, for example colorectal cancer, including, but not limited to metastatic colorectal cancers (mCRC) in a subject comprising: a) obtaining a cancerous tissue sample from the subject; b) contacting the tissue sample with one or more probe sets in the kit of claim 1 or 2; c) assaying the expression of one or more genes from the sample to create a gene signature, wherein principle component analysis is applied to the gene signature to create a probability score for the subject; and d) treating the subject with chemotherapy and bevacizumab (AVASTIN®) when the probability score is high; and treating the subject with chemotherapy and not bevacizumab (AVASTIN®) when the probability score is low. In one aspect, the administration of bevacizumab (AVASTIN®) toa subject with a low probability score has a deleterious effect on the subject. For example, disclosed herein are methods of treating, inhibiting, reducing, decreasing, ameliorating, and/or preventing a cancer and/or metastasis (such as, for example colorectal cancer, including, but not limited to metastatic colorectal cancers (mCRC) in a subject comprising: a) obtaining a cancerous tissue sample from the subject; b) contacting the tissue sample with one or more probe sets that binds to one or more genes that are differentially expressed in the cancer; c) assaying the expression of one or more genes from the sample to create a gene signature, wherein principle component analysis is applied to the gene signature to create a probability score for the subject; and d) treating the subject with chemotherapy and bevacizumab (AVASTIN®) when the probability score is high; and treating the subject with chemotherapy and not bevacizumab (AVASTIN®) when the probability score is low. In one aspect, wherein the subject has a low probability score and is already receiving chemotherapy; bevacizumab is not administered. In one aspect, wherein the subject has a high probability score and is already receiving chemotherapy; bevacizumab is not administered.

Also disclosed herein are methods of treating, inhibiting, reducing, decreasing, ameliorating, and/or preventing a cancer and/or metastasis of any preceding aspect wherein the one or more probe sets comprise merck2_AA101946_x_at, merck2_AA490328_at, merck2_AF129457_at, merck2_AF289590_at, merck2_AI091276_at, merck2_AI223270_at, merck2_AI962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_032_up_at, merck2_VZV_OKA_047_up_at, merck2_XM_938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655 s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_BI045425_at, merck_BI757398_at, merck_BM676784_at, merck_BM805663_at, merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431_s_at, merck_NM_001494_at, merck_NM_001883_at, merck_NM_001993_at, merck_NM_002501_s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41_s_at, merck_VZV_OKA_ORF9_s_at, merck_X85629_at, merck_X98206_at, merck_XM_063314_at merck_XM_210303_at, merck_XM_373233_x_at, merck_XM_378090_at, merck_XM_926011_at, merck_XM_928173_s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, merck_hCT1846664_1_at, merck_hCT2307348_at, or merck_hsa_mir_153_1_x_at

In one aspect, disclosed herein are methods of treating, inhibiting, reducing, decreasing, ameliorating, and/or preventing a cancer and/or metastasis of any preceding aspect, wherein the one or more genes comprise TIMM8A, PRDM15, LOC100130744, LANCL2, LOC101929529, NPHP3 NPHP3-ACAD11, MYO5B, ZSCAN30, ZNF462, CDON. ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725. TISP43 LOC646743, TRIQK, CDH24, LOC101927472, LINC02004, STRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PEL11, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1. In one aspect, the probes bind to at least 1, 2, 3, 4, 5, 6, 7, 8, 9, 10, 11, 12, 13, 14, 15, 16, 17, 18, 19, 20, 21, or 22 of the genes selected from the group consisting of AGR2, BEST4, CRHR2, CRK, CYP24A1, DVL2, ELFN1-AS1, F3, FIP1L1, FOXP1, ICAM2, JAK1, MAP3K14, MBD2, MIB1, MSN, NKX2-5, NODAL, THUMPD3-AS1, TIMELESS, TJP2, and/or USP46. In one aspect, the probes bind to at least 1, 2, 3, 4, 5, 6, 7, 8, 9, 10, 11, 12, 13, or 14 of the genes selected from the group consisting of BFSP2-AS1, ERICH1, HERC4, HIST2H2BF, IWS1, LINC01405, MCHR1, MTMR6, SLC30A2, SLITRK1, SRP68, SRRM4, TFCP2L1, and/or ZNF462. In one aspect, the probes bind to at least 1, 2, 3, 4, 5, 6, 7, or 8 of the genes selected from the group consisting of ERICH1, HERC4, HIST2H2BF. IWS1, MTMR6, SRP68, TFCP2L1, ZNF462 (such as, for example, HERC4, HIST2H2BF, IWS1, SRP68, and/or ZNF462). In one aspect, the probes bind to at least 1, 2, 3, 4, 5, 6, or 7 of the genes selected from the group consisting of HERC4, HIST2H2BF, IWS1, MCHR1, SLC30A2, SRP68, ZNF462. It is understood and herein contemplated that low expression of some genes can be associated with an increase in death (i.e., low survivability and thus high expression is good) including, but not limited to CLPTM1, GTPBP1, IWS1, MAP3K14, MBD2, PIH1D1, SLC25A27, TJP2.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the survival probability for chemotherapy compared to chemotherapy and AVASTIN®.

FIGS. 2A, 2B, 2C, 2D, and 2E show a AVASTIN® efficacy signature by significant Moffitt genes: Moffitt Consortium Cohort: Interaction Model. FIG. 2A shows the overall survival and progression free survival for the AVASTIN® efficacy signature. FIGS. 2B and 2C show signature refined by significant Moffitt genes: Moffitt Cohort: High PC1 for overall survival (2B) and progression free survival (2C). FIG. 2C shows signature by significant Moffitt genes: Moffitt Cohort: Low PC1 for overall survival (2D) and progression free survival (2E).

FIGS. 3A and 3B show validation in the Moffitt consortium cohort. FIG. 3A shows the survival probability for chemotherapy compared to chemotherapy and AVASTIN®. FIG. 3B shows the survival probability for the PC1 high group.

FIGS. 4A, 4B, 4C, and 4D show validation in the Moffitt avatar cohort. FIG. 4A shows the survival probability for chemotherapy compared to chemotherapy and AVASTIN® in the Moffitt avatar cohort. FIG. 4B shows the optimal PC1 Optimal Cutoff Distribution for the avatar cohort. FIG. 4C shows the PC1 Optimal Cutoff (Overall). FIG. 4C shows the PC1 Optimal Cutoff (AVASTIN®).

FIGS. 5A, 5B, and 5C show refined signature based on significant consortium genes for the Moffitt Cohort. FIG. 5A shows the signature refined by significant consortium genes overall. FIG. 5B shows the signature refined by significant consortium genes for High PC1. FIG. 5C shows the signature refined by significant consortium genes for Low PC1.

FIGS. 6A, and 6B show refined signature based on significant consortium genes for the Moffitt Consortium Cohort. FIG. 6A shows the signature refined by significant consortium genes overall. FIG. 6B shows the signature refined by significant consortium genes for High PC1.

FIGS. 7A, and 7B show refined signature based on significant consortium genes for the Moffitt Avatar Cohort. FIG. 7A shows the signature refined by significant consortium genes with a 33% cutoff. FIG. 7B shows the signature refined by significant consortium genes with an optimal PC1 cutoff.

FIGS. 8A, 8B, and 8C show refined signature based on significant avatar genes for the Moffitt Cohort. FIG. 8A shows the signature refined by significant avatar genes overall. FIG. 8B shows the signature refined by significant avatar genes for High PC1. FIG. 8C shows the signature refined by significant avatar genes for Low PC1.

FIGS. 9A, and 9B show refined signature based on significant avatar genes for the Moffitt Consortium Cohort. FIG. 9A shows the signature refined by significant avatar genes overall. FIG. 9B shows the signature refined by significant avatar genes for High PC1.

FIGS. 10A, and 10B show refined signature based on significant avatar genes for the Moffitt Avatar Cohort FIG. 10A shows the signature refined by significant avatar genes for 39% cutoff. FIG. 10B shows the signature refined by significant avatar genes for Optimal PC1 cutoff.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular kit, probe, gene, or gene signature is disclosed and discussed and a number of modifications that can be made to a number of molecules including the kit, probe, gene, or gene signature are discussed, specifically contemplated is each and every combination and permutation of kit, probe, gene, or gene signature and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Hybridization/selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their ka, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 2. Nucleic Acids

There are a variety of molecules disclosed herein that are nucleic acid based. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Primers and Probes

Disclosed in Table 24 are compositions including primers and probes. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

3. Immunoassays and fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow IOGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1, POPO-3; PO-PRO-1; PO-1 PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

### 4. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed herein are kits used for of the treatment of a cancer (such as, for example colorectal cancer, including, but not limited to metastatic colorectal cancers (mCRC) such as, for example, stage 4 mCRC) comprising one or more probe sets comprising merck2_AA101946_x_at, merck2_AA490328_at, merck2_AF129457_at, merck2_AF289590_at, merck2_A1091276_at, merck2_A1223270_at, merck2_A1962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at, merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_032 up_at, merck2_VZV_OKA_047_up_at, merck2_XM_938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655_s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_B1045425_at, merck_BI757398_at, merck_BM676784_at, merck_BM805663_at, merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431_s_at, merck_NM_001494_at, merck_NM_001883_at, merck_NM_001993_at, merck_NM_002501 s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41_s_at, merck_VZV_OKA_ORF9_s_at, merck_X85629_at, merck_X98206_at, merck_XM_063314_at merck_XM_210303_at, merck_XM_373233_x_at, merck_XM_378090_at, merck_XM_926011_at, merck_XM_928173 s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, merck_hCT1846664_1_at, merck_hCT2307348_at, and/or merck_hsa_mir_153_1_x_at

Also disclosed herein are kits, wherein the one or more probe sets bind to one or more genes comprising TIMM8A, PRDM15, LOC100130744, LANCL2, LOC101929529, NPHP3 NPHP3-ACAD11, MYO5B, ZSCAN30, ZNF462, CDON. ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2. SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, and/or MIR153-1.

In one aspect, the kit includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, or 279 of the disclosed probe sets (see for example, Table 24 and those listed above). In one aspect the probe sets bind to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or 198 genes (see for example table 24 and those listed above). In one aspect, the probes bind to at least 1, 2, 3, 4, 5, 6, 7, 8, 9, 10, 11, 12, 13, 14, 15, 16, 17, 18, 19, 20, 21, or 22 of the genes selected from the group consisting of AGR2, BEST4, CRHR2, CRK, CYP24A1, DVL2, ELFN1-AS1, F3, FIP1L1, FOXP1, ICAM2, JAK1, MAP3K14, MBD2, MIB1, MSN, NKX2-5, NODAL, THUMPD3-AS1, TIMELESS, TJP2, and/or USP46. In one aspect, the probes bind to at least 1, 2, 3, 4, 5, 6, 7, 8, 9, 10, 11, 12, 13, or 14 of the genes selected from the group consisting of BFSP2-AS1, ERICH1, HERC4, HIST2H2BF, IWS1, LINC01405, MCHR1, MTMR6, SLC30A2, SLITRK1, SRP68, SRRM4, TFCP2L1, and/or ZNF462. In one aspect, the probes bind to at least 1, 2, 3, 4, 5, 6, 7, or 8 of the genes selected from the group consisting of ERICH1, HERC4, HIST2H2BF, IWS1, MTMR6, SRP68, TFCP2L1, ZNF462 (such as, for example, HERC4, HIST2H2BF, IWS1, SRP68, and/or ZNF462). In one aspect, the probes bind to at least 1, 2, 3, 4, 5, 6, or 7 of the genes selected from the group consisting of HERC4, HIST2H2BF, IWS1, MCHR1, SLC30A2, SRP68, ZNF462. It is understood and herein contemplated that low expression of some genes can be associated with an increase in death (i.e., low survivability and thus high expression is good) including, but not limited to CLPTM1, GTPBP1, IWS1, MAP3K14, MBD2, PIH1D1, SLC25A27, TJP2.

While various embodiments of the present disclosure have been described above, it should be understood that they have been presented by way of example only, and not of limitation. Likewise, the various diagrams may depict an example architectural or other configuration for the invention, which is provided to aid in understanding the features and functionality that can be included in the invention. The invention is not restricted to the illustrated example architectures or configurations, but the desired features can be implemented using a variety of alternative architectures and configurations.

Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

### C. Method of Treating Cancer

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers. In one aspect, the cancer is metastatic colorectal cancer including, but not limited to late term or stage 4 colorectal cancer.

In one aspect, disclosed herein are methods of treating, inhibiting, reducing, decreasing, ameliorating, and/or preventing a cancer and/or metastasis (such as, for example colorectal cancer, including, but not limited to metastatic colorectal cancers (mCRC) in a subject comprising: a) obtaining a cancerous tissue sample from the subject; b) contacting the tissue sample with one or more probesets in the kit of claim 1 or 2; c) assaying the expression of one or more genes from the sample to create a gene signature, wherein principle component analysis is applied to the gene signature to create a probability score for the subject; and d) treating the subject with chemotherapy and bevacizumab (AVASTIN®) when the probability score is high; and treating the subject with chemotherapy and not bevacizumab (AVASTIN®) when the probability score is low. In one aspect, the administration of bevacizumab (AVASTIN®) toa subject with a low probability score has a deleterious effect on the subject. For example, disclosed herein are methods of treating, inhibiting, reducing, decreasing, ameliorating, and/or preventing a cancer and/or metastasis (such as, for example colorectal cancer, including, but not limited to metastatic colorectal cancers (mCRC) in a subject comprising: a) obtaining a cancerous tissue sample from the subject; b) contacting the tissue sample with one or more probesets that binds to one or more genes that are differentially expressed in the cancer; c) assaying the expression of one or more genes from the sample to create a gene signature, wherein principle component analysis is applied to the gene signature to create a probability score for the subject; and d) treating the subject with chemotherapy and bevacizumab (AVASTIN®) when the probability score is high; and treating the subject with chemotherapy and not bevacizumab (AVASTIN®) when the probability score is low. In one aspect, wherein the subject has a low probability score and is already receiving chemotherapy; bevacizumab is not administered. In one aspect, wherein the subject has a high probability score and is already receiving chemotherapy; bevacizumab is not administered.

Also disclosed herein are methods of treating, inhibiting, reducing, decreasing, ameliorating, and/or preventing a cancer and/or metastasis of any preceding aspect wherein the one or more probe sets comprise merck2_AA101946_x_at, merck2_AA490328_at, merck2_AF129457_at, merck2_AF289590_at, merck2_AI091276_at, merck2_AI223270_at, merck2_AI962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_032_up_at, merck2_VZV_OKA_047_up_at, merck2_XM_938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655_s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_BI045425_at, merck_BI757398_at, merck_BM676784_at, merck_BM805663_at merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431 s_at, merck_NM_001494_at, merck_NM_001883_at, merck_NM_001993_at, merck_NM_002501_s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41 s_at, merck_VZV_OKA_ORF9_s_at, merck_X85629_at, merck_X98206_at, merck_XM_063314_at, merck_XM_210303_at, merck_XM_373233_x_at, merck_XM_378090_at, merck_XM_926011_at, merck_XM_928173_s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, merck_hCT1846664_1_at, merck_hCT2307348_at, or merck_hsa_mir_153_1_x_at 79. In one aspect, disclosed herein are methods of treating, inhibiting, reducing, decreasing, ameliorating, and/or preventing a cancer and/or metastasis of any preceding aspect, wherein the one or more genes comprise TIMM8A, PRDM15, LOC100130744, LANCL2, LOC101929529, NPHP3 NPHP3-ACAD11, MYO5B, ZSCAN30, ZNF462, CDON. ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1.

In one aspect, the method includes assaying with at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, or 279 of the disclosed probe sets (see for example, Table 24 and those listed above). In one aspect the probe sets bind to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or 198 genes (see for example table 24 and those listed above). In one aspect, the probes bind to at least 1, 2, 3, 4, 5, 6, 7, 8, 9, 10, 11, 12, 13, 14, 15, 16, 17, 18, 19, 20, 21, or 22 of the genes selected from the group consisting of AGR2, BEST4, CRHR2, CRK, CYP24A1, DVL2, ELFN1-AS1, F3, FIP1L1, FOXP1, ICAM2, JAK1, MAP3K14, MBD2, MIB1, MSN, NKX2-5, NODAL, THUMPD3-AS1, TIMELESS, TJP2, and/or USP46. In one aspect, the probes bind to at least 1, 2, 3, 4, 5, 6, 7, 8, 9, 10, 11, 12, 13, or 14 of the genes selected from the group consisting of BFSP2-AS1, ERICH1, HERC4, HIST2H2BF, IWS1, LINC01405, MCHR1, MTMR6, SLC30A2, SLITRK1, SRP68, SRRM4, TFCP2L1, and/or ZNF462. In one aspect, the probes bind to at least 1, 2, 3, 4, 5, 6, 7, or 8 of the genes selected from the group consisting of ERICH1, HERC4, HIST2H2BF, IWS1, MTMR6, SRP68, TFCP2L1, ZNF462 (such as, for example, HERC4, HIST2H2BF, IWS1, SRP68, and/or ZNF462). In one aspect, the probes bind to at least 1, 2, 3, 4, 5, 6, or 7 of the genes selected from the group consisting of HERC4, HIST2H2BF, IWS1, MCHR1, SLC30A2, SRP68, ZNF462. It is understood and herein contemplated that low expression of some genes can be associated with an increase in death (i.e., low survivability and thus high expression is good) including, but not limited to CLPTM1, GTPBP1, IWS1, MAP3K14, MBD2, PIH1D1, SLC25A27, TJP2.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, AVASTIN® (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeTP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pemnbrolizumab, CT-011, MK-3475), PD-L1 (such as, for example, atezolizum ab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, R07121661), LAG-3 (such as, for example, BMS-986016, LAG525, MK-4280, REGN3767, TSR-033, B1754111, Sym022, FS118, MGD013, and Immutep)

### D. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Development of Predictive AVASTIN® Gene Signature in Colorectal Cancer

An important treatment option for patients diagnosed with metastatic (stage 4) colorectal cancer (mCRC) is an anti-angiogenesis strategy combining chemotherapy with the monoclonal antibody bevacizumab (AVASTIN®) targeting the vascular endothelial growth factor (VEGF). Here, we developed a gene signatures for the potential power in predicting AVASTIN® clinical benefit.

### a) Methods.

Three study cohorts were used to develop the AVASTIN® gene signature: Moffitt training cohort (n=78) for model building, Moffitt Consortium cohort (n=39) and Moffitt Avatar cohort (n=36) as two test sets. Data analysis: Cox proportional hazards method was used to identify predictive AVASTIN® gene signatures by utilizing a statistical interaction term of gene signature score and treatment in the model. Principal component analysis (PCA) was used for data reduction to derive a summary predictive score.

### b) Results.

Univariate Cox analysis of the interaction model yielded 279 probe sets (198 unique genes) associated with AVASTIN® effect. Analysis of the median cutoff of the first principal component of the 279 probe sets showed patients treated with AVASTIN® tended to improve survival for high PC1 score group (p<0.001). On the other hand, a patient with a low PC1 score seems to have better OS if treated with chemotherapy only (p<0.001). In Consortium cohort, the signature showed numerically improved median OS if AVASTIN®+Chemo was given (p=0.1). For the Avatar cohort, the median OS was higher in high PC1 compared to low PC1 in the overall group (p=0.04) and in the AVASTIN®+Chemo group (p=0.11). In the refinement analysis of AVASTIN® gene signature, the Consortium significant genes (14 genes) showed robust predictive effect in the three cohorts. The Avatar significant genes (8 genes) showed robust predictive effect (prediction of AVASTIN®) in Moffitt training cohort and also showed numerically improved OS for patients with high PC1 in overall and in the AVASTIN® group.

### (1) Moffit Training Cohort

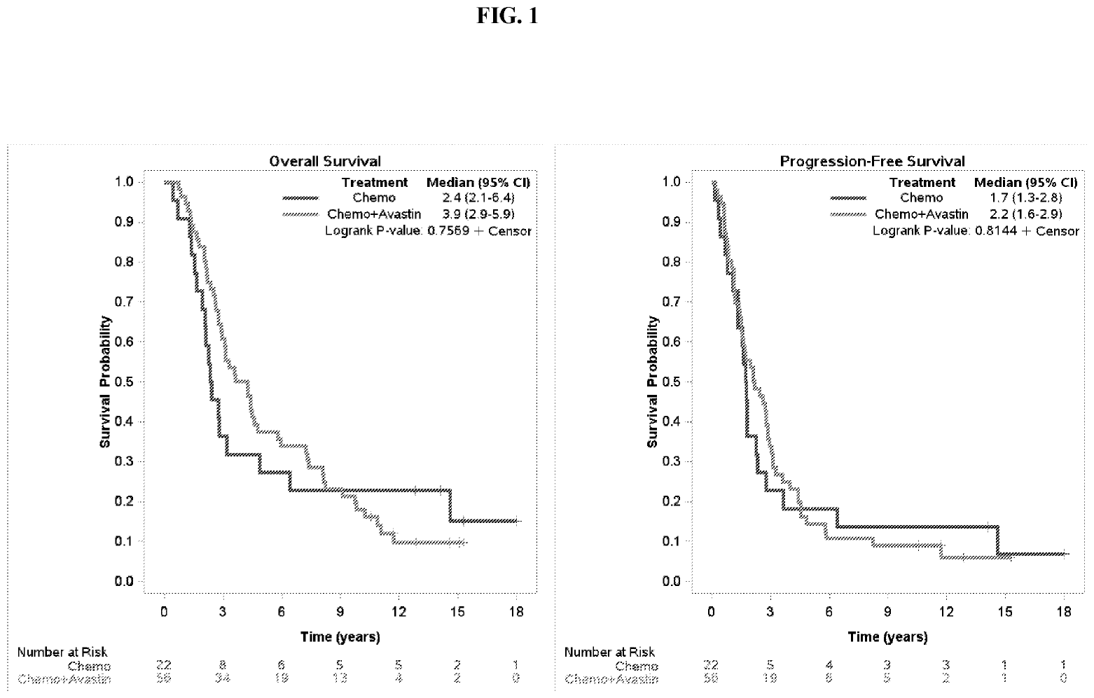

To investigate the efficacy of AVASTIN, to treat metastatic colorectal cancer (mCRC) patients, we looked at gene expression within a population of 78 patients with mCRC. Among the cohort, the overall survival was incredibly low with 87% (68 patients) dying from CRC. In this population 56 patients (72%) received AVASTIN® and chemotherapy and the remaining 22 patients (28%) only received chemotherapy. Gene expression data were generated using HuRSTA-2a520709 Affymetrix arrays and normalized by the iterative rank order normalization method. As shown in the comparison of overall survival and progression free survival between the two treatment groups did not yield a significant AVASTIN® treatment effect (p=0.79; FIG. 1 and Table 1). While the median OS was 4.26 years in patients treated with AVASTIN®+chemotherapy versus 2.58 years in patients with chemotherapy only, the survival curves started to crossover at year 9 (FIG. 1).

TABLE 1
Median Survival Time of Treatment in Moffitt Cohort

| strata | median | lower | upper |
|---|---|---|---|
| Treatment = Chemo | 2.579452 | 2.079452 | 6.416438 |
| Treatment = Chemo + AVASTIN ® | 4.257534 | 3.093151 | 5.934247 |

### (2) Model Building Using Interaction Model to Predict AVASTIN® Effect

Wanting to find a way to distinguish patients that would respond favorably to AVASTIN®, we used an interaction model to predict AVASTIN® effect. Cox proportional hazards method was used to develop a predictive AVASTIN® gene signature. Specifically, a statistical interaction model for prediction of treatment benefit was built including two variables, treatment and a binary probe set expression, as main effects, and an interaction term of both variables. The binary probe set expression variable was constructed based on median cutoff of the probe set expression. Principal component analysis (PCA) was performed to summarize the significant genes.

### (a) Univariate Analysis

Results showed 279 probe sets with adjusted p value<0.05 in the interaction term. These probe sets were linked to 198 genes (See Table 24). Among these significant probe sets, there were 152 probe sets with main effect of HR>1 for patient with chemotherapy only (median HR=7.93; interquartile range: 6.82-9.95). In other words, higher expression of these probe sets increased death risk if patients were not treated with AVASTIN®. On the other hand, the HR in the interaction term became less than 1 for patients additionally treated with AVASTIN® (median HR=0.09; interquartile range: 0.08-0.1). This indicates that AVASTIN® could improve OS for patients with high expression of these probe sets. The other 127 probe sets had opposite trend with HR<1 in the main effect (median HR=0.12; interquartile range: 0.1-0.14) and HR>1 in the interaction term (median HR=10.63; interquartile range: 9.79-12.72), showing association of low expression with high risk for patients treated with chemotherapy only and with improved OS when AVASTIN® was received.

| | | | | | | | | | | | | Probe | |
|---|---|---|---|---|---|---|---|---|---|---|---|---|---|
| | | | | Probe set | | | | Treatment | | | | set*Treatment | |
| ProbeID | Symbol | HR (95%) | | p_value | adj_pvalue | HR (95%) | | p_value | adj_pvalue | HR (95%) | | p_value | adj_pvalue |
| merck2_AA101946_x_at | TIMM8A | 0.09 | (0.03-0.27) | 1.51E−05 | 0.02735 | 0.26 | (0.13-0.53) | 2.40E−04 | 0.049330742 | 10.46 | (3.07-35.59) | 1.72E−04 | 0.043915 |
| merck2_AA490328_at | PRDM15 | 0.12 | (0.04-0.33) | 3.52E−05 | 0.03162 | 0.2 | (0.09-0.46) | 1.06E−04 | 0.037518224 | 9.87 | (3.09-31.56) | 1.12E−04 | 0.037908 |
| merck2_AF129457_at | — | 0.14 | (0.05-0.38) | 1.09E−04 | 0.0378 | 0.19 | (0.08-0.43) | 8.79E−05 | 0.037355406 | 11.41 | (3.52-36.98) | 4.91E−05 | 0.033119 |
| merck2_AF289590_at | LOC100130744 | 0.13 | (0.05-0.36) | 8.13E−05 | 0.03674 | 0.24 | (0.11-0.51) | 1.97E−04 | 0.046260143 | 9.44 | (2.99-29.81) | 1.29E−04 | 0.040085 |
| merck2_AI091276_at | LANCL2 | 0.08 | (0.03-0.26) | 2.33E−05 | 0.02905 | 0.24 | (0.11-0.49) | 9.22E−05 | 0.037355406 | 16.15 | (4.34-60.04) | 3.30E−05 | 0.031623 |
| merck2_AI223270_at | LOC101929529 | 3.6 | (1.34-9.63) | 0.010925 | 0.21429 | 2.38 | (1.13-5.01) | 0.02234 | 0.288955919 | 0.11 | (0.03-0.35) | 2.06E−04 | 0.046856 |
| merck2_AI962276_at | — | 10.26 | (2.93-35.92) | 2.71E−04 | 0.05071 | 5.38 | (1.63-17.78) | 0.00583 | 0.165007322 | 0.05 | (0.01-0.19) | 1.51E−05 | 0.027346 |
| merck2_AK092910_at | NPHP3 | 8.55 | (3.16-23.12) | 2.38E−05 | 0.02905 | 2.22 | (0.95-5.19) | 0.06629 | 0.442914315 | 0.1 | (0.03-0.32) | 9.21E−05 | 0.037355 |
| | NPHP3- | | | | | | | | | | | | |
| | ACAD11 | | | | | | | | | | | | |
| merck2_AK093149_at | MYO5B | 8.51 | (3.02-23.95) | 5.05E−05 | 0.03312 | 1.95 | (0.92-4.14) | 0.08206 | 0.478296378 | 0.08 | (0.02-0.26) | 2.97E−05 | 0.030943 |
| merck2_AK131291_at | ZSCAN30 | 6.61 | (2.35-18.55) | 3.40E−04 | 0.05425 | 3.19 | (1.28-7.92) | 0.01258 | 0.227301814 | 0.08 | (0.02-0.25) | 2.75E−05 | 0.030721 |
| merck2_AL359561_at | ZNF462 | 6.19 | (2.18-17.54) | 6.08E−04 | 0.06881 | 2.93 | (1.12-7.65) | 0.02815 | 0.318119551 | 0.1 | (0.03-0.34) | 1.99E−04 | 0.046293 |
| merck2_AL832223_at | CDON | 7.91 | (2.82-22.17) | 8.53E−05 | 0.03698 | 2.58 | (1.05-6.34) | 0.03845 | 0.359052143 | 0.1 | (0.03-0.32) | 1.11E−04 | 0.037864 |
| merck2_AL834299_at | ZNF689 | 0.14 | (0.05-0.39) | 1.13E−04 | 0.03791 | 0.22 | (0.1-0.49) | 1.62E−04 | 0.042632345 | 9.82 | (3.09-31.2) | 1.08E−04 | 0.03757 |
| merck2_AL834442_at | NCOA7 | 8.06 | (2.92-22.23) | 5.51E−05 | 0.03338 | 1.71 | (0.8-3.65) | 0.16339 | 0.607643412 | 0.1 | (0.03-0.33) | 1.35E−04 | 0.040323 |
| merck2_AM392650_at | ZNF280D | 6.39 | (2.35-17.34) | 2.72E−04 | 0.05071 | 2.66 | (1.12-6.27) | 0.02586 | 0.307359543 | 0.09 | (0.03-0.29) | 6.18E−05 | 0.034824 |
| merck2_AW007481_at | NFIX | 0.1 | (0.03-0.34) | 1.57E−04 | 0.04236 | 0.26 | (0.13-0.54) | 2.82E−04 | 0.051044945 | 15.15 | (3.99-57.49) | 6.46E−05 | 0.034824 |
| merck2_AX721105_at | ZNF467 | 9.82 | (3.48-27.69) | 1.58E−05 | 0.02735 | 2.04 | (0.88-4.75) | 0.09651 | 0.507347422 | 0.11 | (0.03-0.35) | 1.85E−04 | 0.045272 |
| merck2_BC001038_at | EPN3 | 7.53 | (2.73-20.78) | 9.79E−05 | 0.03736 | 1.83 | (0.87-3.86) | 0.11237 | 0.535814749 | 0.1 | (0.03-0.31) | 8.20E−05 | 0.036737 |
| merck2_BC033091_at | SLC25A27 | 7.4 | (2.49-21.93) | 3.08E−04 | 0.05243 | 3.15 | (1.2-8.29) | 0.02008 | 0.277414194 | 0.09 | (0.03-0.3) | 1.07E−04 | 0.03757 |
| merck2_BC035840_at | GJC2 | 0.13 | (0.05-0.34) | 3.67E−05 | 0.03162 | 0.2 | (0.09-0.44) | 5.64E−05 | 0.033692775 | 10.97 | (3.47-34.71) | 4.61E−05 | 0.033064 |
| merck2_BC039861_at | NODAL | 0.12 | (0.04-0.34) | 6.27E−05 | 0.03482 | 0.24 | (0.11-0.49) | 1.25E−04 | 0.039651341 | 11.69 | (3.55-38.46) | 5.18E−05 | 0.033119 |
| merck2_BE249972_x_at | RPL17 | 4.19 | (1.6-10.98) | 0.003494 | 0.13421 | 2.3 | (1.05-5.05) | 0.03744 | 0.355433593 | 0.12 | (0.04-0.37) | 2.44E−04 | 0.049436 |
| | RPL17- | | | | | | | | | | | | |
| | C18orf32 | | | | | | | | | | | | |
| merck2_BE349054_x_at | TDRKH | 6.26 | (2.31-16.97) | 3.12E−04 | 0.05259 | 2.52 | (1.08-5.91) | 0.03313 | 0.338695291 | 0.1 | (0.03-0.32) | 9.93E−05 | 0.037355 |
| merck2_BF248252_at | FAM149B1 | 7.56 | (2.54-22.52) | 2.82E−04 | 0.05104 | 3.08 | (1.16-8.2) | 0.02421 | 0.29951072 | 0.09 | (0.03-0.32) | 1.87E−04 | 0.045541 |
| merck2_BF591243_at | PPIE | 0.12 | (0.04-0.32) | 4.02E−05 | 0.03191 | 0.13 | (0.05-0.32) | 1.20E−05 | 0.027345536 | 16.14 | (4.89-53.32) | 5.08E−06 | 0.027253 |
| merck2_BF692777_at | — | 0.14 | (0.05-0.37) | 1.02E−04 | 0.03736 | 0.19 | (0.08-0.44) | 1.42E−04 | 0.040831643 | 9.22 | (2.88-29.51) | 1.81E−04 | 0.044798 |
| merck2_BG896934_at | FIP1L1 | 0.13 | (0.04-0.35) | 9.16E−05 | 0.03736 | 0.2 | (0.09-0.43) | 3.12E−05 | 0.031476456 | 17.67 | (5.2-60.11) | 4.25E−06 | 0.027253 |
| merck2_BI047264_at | — | 0.14 | (0.05-0.36) | 6.54E−05 | 0.03482 | 0.22 | (0.1-0.48) | 1.28E−04 | 0.040085056 | 9.54 | (3.04-29.94) | 1.10E−04 | 0.037864 |
| merck2_BM545067_s_at | MRPL38 | 0.17 | (0.06-0.47) | 5.96E−04 | 0.06831 | 0.27 | (0.13-0.57) | 6.32E−04 | 0.069813311 | 9.01 | (2.83-28.72) | 2.02E−04 | 0.046589 |
| merck2_BM681401_at | GABPB1-AS1 | 6.83 | (2.4-19.47) | 3.23E−04 | 0.05346 | 1.58 | (0.78-3.22) | 0.20789 | 0.657976999 | 0.11 | (0.03-0.35) | 2.40E−04 | 0.049331 |
| merck2_BU618242_at | GALNT1 | 10.19 | (3.59-28.97) | 1.33E−05 | 0.02735 | 2.56 | (1.03-6.31) | 0.04196 | 0.371529111 | 0.09 | (0.03-0.29) | 6.51E−05 | 0.034824 |
| merck2_BX115227_at | ERICH1 | 0.18 | (0.06-0.52) | 0.001404 | 0.09482 | 0.3 | (0.15-0.61) | 8.84E−04 | 0.07968467 | 10.48 | (3.16-34.74) | 1.22E−04 | 0.039022 |
| merck2_BX647415_at | CEP126 | 8.69 | (2.95-25.66) | 8.97E−05 | 0.03736 | 2.97 | (1.13-7.8) | 0.02721 | 0.314222724 | 0.09 | (0.03-0.31) | 1.19E−04 | 0.038671 |
| merck2_BX647735_at | AP3S1 | 9.91 | (3.33-29.52) | 3.79E−05 | 0.03163 | 2.86 | (1.09-7.56) | 0.03353 | 0.340363806 | 0.09 | (0.03-0.31) | 1.51E−04 | 0.041766 |
| merck2_BX648311_at | AHI1 | 8.53 | (3.11-23.4) | 3.08E−05 | 0.03148 | 2.56 | (1.09-6.01) | 0.03106 | 0.330098208 | 0.08 | (0.02-0.26) | 2.26E−05 | 0.029051 |
| merck2_BY799718_at | MRPL1 | 7.72 | (2.39-24.96) | 6.37E−04 | 0.06996 | 4.09 | (1.39-12.05) | 0.01061 | 0.211292893 | 0.07 | (0.02-0.27) | 9.05E−05 | 0.037355 |
| merck2_CMV_UL43_at | — | 0.13 | (0.04-0.36) | 9.16E−05 | 0.03736 | 0.26 | (0.13-0.55) | 4.19E−04 | 0.058762307 | 9.49 | (2.86-31.46) | 2.32E−04 | 0.048678 |
| merck2_CR749241_s_at | FRMPD2 | 5.33 | (2.02-14.08) | 7.23E−04 | 0.07378 | 2.15 | (0.99-4.68) | 0.05442 | 0.41093829 | 0.11 | (0.04-0.34) | 1.45E−04 | 0.041504 |
| merck2_CX783886_a_at | UBE3A | 0.15 | (0.05-0.41) | 2.16E−04 | 0.0473 | 0.18 | (0.07-0.44) | 1.59E−04 | 0.042549339 | 9.65 | (2.99-31.13) | 1.49E−04 | 0.041686 |
| merck2_DB492896_at | ZNF365 | 0.14 | (0.05-0.39) | 1.42E−04 | 0.04083 | 0.18 | (0.07-0.43) | 1.33E−04 | 0.04014514 | 9.35 | (2.91-30.01) | 1.72E−04 | 0.043915 |
| merck2_DQ891843_at | ICAM2 | 0.16 | (0.06-0.44) | 4.41E−04 | 0.0597 | 0.28 | (0.14-0.58) | 6.75E−04 | 0.071481634 | 9.39 | (2.89-30.54) | 1.98E−04 | 0.04626 |
| merck2_{—} | PRR21 | 7.04 | (2.59-19.15) | 1.33E−04 | 0.04015 | 2.51 | (1.07-5.88) | 0.03383 | 0.341840876 | 0.09 | (0.03-0.29) | 6.05E−05 | 0.034824 |
| ENST00000355104_x_at | | | | | | | | | | | | | |
| merck2_{—} | CT47A1 | 8.54 | (2.64-27.62) | 3.42E−04 | 0.05425 | 4.09 | (1.39-12.05) | 0.01068 | 0.21164556 | 0.07 | (0.02-0.25) | 7.02E−05 | 0.034828 |
| ENST00000371290_s_at | CT47A10 | | | | | | | | | | | | |
| | CT47A11 | | | | | | | | | | | | |
| | CT47A12 | | | | | | | | | | | | |
| | CT47A2 | | | | | | | | | | | | |
| | CT47A3 | | | | | | | | | | | | |
| | CT47A4 | | | | | | | | | | | | |
| | CT47A5 | | | | | | | | | | | | |
| | CT47A6 | | | | | | | | | | | | |
| | CT47A7 | | | | | | | | | | | | |
| | CT47A8 | | | | | | | | | | | | |
| | CT47A9 | | | | | | | | | | | | |
| merck2_F25718_at | LINC01405 | 5.34 | (2.01-14.21) | 7.86E−04 | 0.07573 | 1.96 | (0.92-4.16) | 0.08171 | 0.477550212 | 0.12 | (0.04-0.36) | 2.29E−04 | 0.048335 |
| merck2_HSV1_ORF_UL46_at | — | 0.08 | (0.02-0.28) | 9.52E−05 | 0.03736 | 0.25 | (0.12-0.5) | 1.03E−04 | 0.037511443 | 21.46 | (5.17-89.11) | 2.43E−05 | 0.029291 |
| merck2_HSV2_ORF_UL18_at | — | 0.1 | (0.03-0.27) | 7.14E−06 | 0.02725 | 0.19 | (0.09-0.43) | 7.43E−05 | 0.035381836 | 9.25 | (2.89-29.57) | 1.77E−04 | 0.044526 |
| merck2_JC_vir_smTAg_at | — | 0.16 | (0.06-0.42) | 2.44E−04 | 0.04944 | 0.26 | (0.12-0.56) | 4.81E−04 | 0.061918193 | 8.77 | (2.75-28.01) | 2.47E−04 | 0.049436 |
| merck2_NM_001005224_s_at | OR4F16 | 6.16 | (2.33-16.28) | 2.47E−04 | 0.04944 | 2.15 | (1-4.6) | 0.04865 | 0.393922587 | 0.09 | (0.03-0.28) | 3.98E−05 | 0.031905 |
| | OR4F21 | | | | | | | | | | | | |
| | OR4F29 | | | | | | | | | | | | |
| | OR4F3 | | | | | | | | | | | | |
| merck2_NM_001037866_s_at | RGPD5 | 10.63 | (3.53-31.97) | 2.61E−05 | 0.03021 | 3.45 | (1.3-9.15) | 0.01261 | 0.227520734 | 0.06 | (0.02-0.21) | 1.26E−05 | 0.027346 |
| | RGPD6 | | | | | | | | | | | | |
| merck2_NM_001080537_at | SNTN | 6.65 | (2.44-18.16) | 2.17E−04 | 0.04746 | 2.35 | (1.01-5.5) | 0.04813 | 0.392153506 | 0.11 | (0.03-0.34) | 1.61E−04 | 0.04262 |
| merck2_NM_005570_at | LMAN1 | 11.29 | (3.81-33.43) | 1.20E−05 | 0.02735 | 2.78 | (1.06-7.33) | 0.03801 | 0.35718887 | 0.09 | (0.03-0.3) | 1.09E−04 | 0.037798 |
| merck2_NM_006485_at | FBLN1 | 0.12 | (0.04-0.33) | 5.44E−05 | 0.03322 | 0.22 | (0.1-0.46) | 7.44E−05 | 0.035381836 | 13.28 | (3.98-44.31) | 2.61E−05 | 0.030212 |
| merck2_NM_006594_at | AP4B1 | 7.04 | (2.66-18.6) | 8.37E−05 | 0.03698 | 2.21 | (1.01-4.83) | 0.04674 | 0.387728354 | 0.09 | (0.03-0.27) | 2.48E−05 | 0.029623 |
| merck2_NM_022079_at | HERC4 | 9.26 | (3.1-27.65) | 6.61E−05 | 0.03482 | 2.84 | (1.08-7.51) | 0.03503 | 0.34676014 | 0.1 | (0.03-0.33) | 2.13E−04 | 0.047264 |
| merck2_NM_022747_at | CCDC85C | 0.13 | (0.05-0.36) | 7.38E−05 | 0.03538 | 0.21 | (0.1-0.47) | 1.52E−04 | 0.041766283 | 9.83 | (3.06-31.53) | 1.22E−04 | 0.039022 |
| merck2_NM_025214_at | CCDC68 | 6.36 | (2.25-17.95) | 4.81E−04 | 0.06192 | 2.94 | (1.18-7.31) | 0.02005 | 0.27727444 | 0.09 | (0.03-0.3) | 8.72E−05 | 0.037305 |
| merck2_NM_030813_at | CLPB | 0.24 | (0.09-0.65) | 0.005245 | 0.15777 | 0.29 | (0.14-0.62) | 0.00133 | 0.092706923 | 9.77 | (3.02-31.63) | 1.42E−04 | 0.040832 |
| merck2_NM_032260_s_at | RGPD5 | 9.17 | (3.33-25.24) | 1.81E−05 | 0.02848 | 2.57 | (1.1-6.01) | 0.02965 | 0.324340319 | 0.07 | (0.02-0.24) | 1.32E−05 | 0.027346 |
| | RGPD6 | | | | | | | | | | | | |
| merck2_NM_032639_at | PLEKHA8 | 6.94 | (2.26-21.31) | 7.18E−04 | 0.0737 | 3.53 | (1.23-10.15) | 0.01913 | 0.271891726 | 0.09 | (0.02-0.31) | 1.74E−04 | 0.044177 |
| merck2_NM_052996_a_at | PRDM7 | 7.48 | (2.67-20.96) | 1.28E−04 | 0.04009 | 2.68 | (1.09-6.61) | 0.03226 | 0.334917286 | 0.1 | (0.03-0.31) | 1.02E−04 | 0.037435 |
| merck2_NM_205833_at | IGSF1 | 0.1 | (0.04-0.26) | 5.36E−06 | 0.02725 | 0.2 | (0.1-0.44) | 4.15E−05 | 0.032528361 | 10.24 | (3.25-32.27) | 7.18E−05 | 0.034986 |
| merck2_VZV_OKA_O32_up_at | — | 14.48 | (4.49-46.66) | 7.55E−06 | 0.02725 | 4.06 | (1.41-11.73) | 0.0095 | 0.201373437 | 0.05 | (0.01-0.18) | 6.62E−06 | 0.027253 |
| merck2_VZV_OKA_O47_up_at | — | 0.13 | (0.05-0.36) | 9.59E−05 | 0.03736 | 0.26 | (0.12-0.54) | 3.12E−04 | 0.052591683 | 10.14 | (3.06-33.64) | 1.53E−04 | 0.041846 |
| merck2_XM_938193_at | SH2D1A | 0.18 | (0.07-0.48) | 7.18E−04 | 0.0737 | 0.24 | (0.11-0.51) | 2.32E−04 | 0.048677914 | 12.5 | (3.82-40.9) | 2.96E−05 | 0.030943 |
| | STAG2 | | | | | | | | | | | | |
| | HSPA8P20 | | | | | | | | | | | | |
| | NPM1P34 | | | | | | | | | | | | |
| | TEX13D | | | | | | | | | | | | |
| | ZIK1P1 | | | | | | | | | | | | |
| | LOC101928402 | | | | | | | | | | | | |
| merck_AB058756_a_at | SRRM4 | 11.07 | (3.8-32.27) | 1.07E−05 | 0.02735 | 1.69 | (0.81-3.52) | 0.15845 | 0.601270337 | 0.07 | (0.02-0.23) | 1.88E−05 | 0.028478 |
| merck_AF038451_a_at | AGR2 | 8.28 | (2.81-24.46) | 1.30E−04 | 0.04009 | 2.85 | (1.08-7.5) | 0.03408 | 0.342860298 | 0.1 | (0.03-0.34) | 2.47E−04 | 0.049436 |
| merck_AF085981_at | ENTPD4 | 10.97 | (3.96-30.38) | 4.01E−06 | 0.02725 | 2.04 | (0.93-4.46) | 0.07442 | 0.460959791 | 0.07 | (0.02-0.22) | 7.63E−06 | 0.027253 |
| merck_AF118083_a_at | EXOC5 | 7.93 | (2.68-23.45) | 1.80E−04 | 0.0448 | 2.94 | (1.11-7.79) | 0.03039 | 0.327726409 | 0.1 | (0.03-0.34) | 2.35E−04 | 0.0488 |
| merck_AF143326_at | FOXP1 | 7.83 | (2.92-20.97) | 4.27E−05 | 0.03286 | 2.45 | (1.09-5.53) | 0.03058 | 0.328203492 | 0.08 | (0.02-0.24) | 1.33E−05 | 0.027346 |
| | FOXP1-AS1 | | | | | | | | | | | | |
| | MIR1284 | | | | | | | | | | | | |
| merck_AF147412_at | ARID1B | 7.1 | (2.52-20.03) | 2.12E−04 | 0.04726 | 2.93 | (1.19-7.24) | 0.01969 | 0.275255651 | 0.08 | (0.03-0.27) | 4.57E−05 | 0.033064 |
| | MIR4466 | | | | | | | | | | | | |
| merck_AF147431_at | DIS3L | 7.53 | (2.66-21.33) | 1.46E−04 | 0.0415 | 2.99 | (1.2-7.44) | 0.01838 | 0.268016286 | 0.08 | (0.02-0.26) | 3.88E−05 | 0.031887 |
| merck_AF161340_at | — | 0.1 | (0.03-0.28) | 2.43E−05 | 0.02929 | 0.27 | (0.13-0.56) | 4.02E−04 | 0.057697091 | 9.81 | (2.89-33.23) | 2.47E−04 | 0.049436 |
| merck_AF169158_a_at | CABP5 | 7.26 | (2.68-19.69) | 9.90E−05 | 0.03736 | 2.54 | (1.08-5.96) | 0.03208 | 0.334214231 | 0.09 | (0.03-0.28) | 4.75E−05 | 0.033119 |
| merck_AF311308_at | MGAT1 | 0.11 | (0.04-0.3) | 1.36E−05 | 0.02735 | 0.2 | (0.09-0.43) | 5.17E−05 | 0.033119082 | 9.95 | (3.18-31.12) | 7.82E−05 | 0.036384 |
| merck_AI204893_at | — | 0.16 | (0.06-0.42) | 2.79E−04 | 0.05083 | 0.25 | (0.12-0.53) | 3.04E−04 | 0.052173629 | 10.1 | (3.14-32.53) | 1.06E−04 | 0.037518 |
| merck_AJ406951_s_at | KRTAP9-9 | 0.12 | (0.04-0.33) | 3.78E−05 | 0.03163 | 0.21 | (0.1-0.45) | 6.82E−05 | 0.034824234 | 11.47 | (3.58-36.77) | 4.00E−05 | 0.031905 |
| merck_AJ459838_at | OR2H1 | 10.99 | (4-30.19) | 3.36E−06 | 0.02725 | 2.67 | (1.18-6) | 0.01793 | 0.264995899 | 0.05 | (0.02-0.17) | 7.21E−07 | 0.023449 |
| merck_AK000932_at | — | 5.04 | (1.88-13.46) | 0.00127 | 0.09185 | 2.39 | (1.06-5.38) | 0.03526 | 0.347755016 | 0.11 | (0.03-0.34) | 1.60E−04 | 0.04262 |
| merck_AK021682_at | COL9A2 | 6.82 | (2.58-17.98) | 1.05E−04 | 0.03752 | 1.85 | (0.85-4.02) | 0.12078 | 0.548746405 | 0.12 | (0.04-0.37) | 2.04E−04 | 0.046691 |
| merck_AK022168_at | CSTF2T | 13.73 | (3.78-49.91) | 6.95E−05 | 0.03482 | 4.5 | (1.35-15.03) | 0.01452 | 0.241098101 | 0.06 | (0.02-0.26) | 1.38E−04 | 0.040515 |
| | MIR605 | | | | | | | | | | | | |
| | PRKG1 | | | | | | | | | | | | |
| | RSU1P3 | | | | | | | | | | | | |
| | LOC102724719 | | | | | | | | | | | | |
| merck_AK022347_at | CSTF2T | 9.46 | (2.96-30.23) | 1.52E−04 | 0.04177 | 3.38 | (1.17-9.78) | 0.02458 | 0.30104221 | 0.09 | (0.03-0.33) | 2.51E−04 | 0.049663 |
| | MIR605 | | | | | | | | | | | | |
| | PRKG1 | | | | | | | | | | | | |
| | RSU1P3 | | | | | | | | | | | | |
| | LOC102724719 | | | | | | | | | | | | |
| merck_AK022443_at | FOXP1 | 8.11 | (2.98-22.07) | 4.25E−05 | 0.03285 | 2.27 | (0.97-5.33) | 0.05906 | 0.423367826 | 0.1 | (0.03-0.32) | 1.03E−04 | 0.037435 |
| | FOXP1-AS1 | | | | | | | | | | | | |
| | MIR1284 | | | | | | | | | | | | |
| merck_AK023601_at | EFR3A | 4.64 | (1.64-13.08) | 0.00377 | 0.13795 | 3.42 | (1.37-8.56) | 0.00857 | 0.193122405 | 0.08 | (0.02-0.28) | 6.27E−05 | 0.034824 |
| merck_AK024198_at | — | 5.33 | (1.88-15.1) | 0.001665 | 0.10174 | 2.9 | (1.16-7.25) | 0.02263 | 0.29077884 | 0.1 | (0.03-0.34) | 2.19E−04 | 0.047534 |
| merck_AK024231_at | — | 7.92 | (2.64-23.79) | 2.27E−04 | 0.0481 | 4.2 | (1.56-11.31) | 0.00454 | 0.148604076 | 0.05 | (0.01-0.19) | 6.61E−06 | 0.027253 |
| merck_AK024810_a_at | PRPF40A | 10.36 | (3.51-30.59) | 2.34E−05 | 0.02905 | 2.73 | (1.04-7.18) | 0.04141 | 0.369596922 | 0.1 | (0.03-0.33) | 1.74E−04 | 0.044237 |
| merck_AK025202_a_at | LARGE1 | 6.19 | (2.32-16.51) | 2.68E−04 | 0.05048 | 2.22 | (0.99-4.99) | 0.05327 | 0.407683841 | 0.11 | (0.03-0.34) | 1.23E−04 | 0.039214 |
| merck_AK025621_at | LOC101927365 | 6.56 | (2.42-17.77) | 2.13E−04 | 0.04726 | 2.27 | (0.97-5.31) | 0.05823 | 0.421379596 | 0.11 | (0.04-0.36) | 2.34E−04 | 0.0488 |
| merck_AK026808_at | HERC4 | 10.59 | (3.76-29.82) | 8.00E−06 | 0.02725 | 2.25 | (0.92-5.53) | 0.07673 | 0.466503261 | 0.11 | (0.03-0.35) | 2.02E−04 | 0.046589 |
| merck_AK054931_at | MIR9-3HG | 0.18 | (0.07-0.48) | 6.39E−04 | 0.06996 | 0.28 | (0.13-0.58) | 6.39E−04 | 0.069958172 | 8.83 | (2.81-27.75) | 1.94E−04 | 0.046237 |
| merck_AK055172_a_at | DLG5 | 0.13 | (0.05-0.36) | 7.40E−05 | 0.03538 | 0.22 | (0.1-0.48) | 1.59E−04 | 0.042549339 | 9.55 | (3-30.39) | 1.33E−04 | 0.040145 |
| merck_AK055584_at | STT3B | 10.98 | (3.43-35.17) | 5.50E−05 | 0.03338 | 3.67 | (1.26-10.69) | 0.01712 | 0.258886743 | 0.07 | (0.02-0.27) | 9.11E−05 | 0.037355 |
| merck_AK091999_at | LOC101929713 | 7.57 | (2.81-20.38) | 6.25E−05 | 0.03482 | 2.19 | (0.94-5.1) | 0.06933 | 0.449887537 | 0.11 | (0.04-0.35) | 1.60E−04 | 0.04262 |
| merck_AK093735_s_at | CEP83 | 5.78 | (2.2-15.19) | 3.68E−04 | 0.05564 | 2.14 | (0.98-4.68) | 0.0558 | 0.414908314 | 0.11 | (0.03-0.33) | 1.06E−04 | 0.037518 |
| merck_AK095449_at | CRK | 11.71 | (3.91-35.06) | 1.10E−05 | 0.02735 | 3.33 | (1.26-8.82) | 0.01551 | 0.248286757 | 0.06 | (0.02-0.22) | 1.37E−05 | 0.027346 |
| merck_AK096712_at | USP46 | 7.5 | (2.64-21.27) | 1.51E−04 | 0.04177 | 2.87 | (1.16-7.09) | 0.02235 | 0.288987713 | 0.08 | (0.03-0.28) | 5.28E−05 | 0.033119 |
| merck_AK097571_s_at | LOC105378730 | 0.13 | (0.05-0.37) | 8.91E−05 | 0.03736 | 0.22 | (0.1-0.48) | 1.08E−04 | 0.037570373 | 11.45 | (3.55-36.87) | 4.42E−05 | 0.033064 |
| merck_AK097840_a_at | LOC105379589 | 0.15 | (0.05-0.41) | 2.33E−04 | 0.0487 | 0.26 | (0.13-0.54) | 3.11E−04 | 0.052591683 | 10.5 | (3.24-34.1) | 9.07E−05 | 0.037355 |
| | LOC107984097 | | | | | | | | | | | | |
| | LOC107987409 | | | | | | | | | | | | |
| | LOC107987410 | | | | | | | | | | | | |
| merck_AK123535_at | FBXL18 | 0.12 | (0.05-0.33) | 3.00E−05 | 0.03094 | 0.19 | (0.08-0.4) | 2.33E−05 | 0.029051464 | 12.87 | (4.08-40.62) | 1.31E−05 | 0.027346 |
| merck_AK123655_s_at | UCHL5 | 4.51 | (1.7-12.02) | 0.002561 | 0.11983 | 2.52 | (1.11-5.71) | 0.02708 | 0.313519552 | 0.11 | (0.03-0.35) | 1.64E−04 | 0.042845 |
| merck_AK123888_at | ZNF555 | 12.35 | (4.32-35.34) | 2.76E−06 | 0.02725 | 2.14 | (0.92-4.98) | 0.07815 | 0.470065199 | 0.08 | (0.03-0.27) | 3.54E−05 | 0.031623 |
| merck_AK125149_at | CD99P1 | 9.95 | (3.56-27.78) | 1.15E−05 | 0.02735 | 1.87 | (0.86-4.07) | 0.11536 | 0.540645886 | 0.09 | (0.03-0.27) | 3.52E−05 | 0.031623 |
| merck_AK127206_s_at | LONRF2 | 0.1 | (0.04-0.28) | 9.11E−06 | 0.02735 | 0.18 | (0.08-0.41) | 3.53E−05 | 0.031623068 | 10.83 | (3.41-34.41) | 5.36E−05 | 0.033119 |
| merck_AK130508_at | LOC100507281 | 7.15 | (2.65-19.28) | 1.02E−04 | 0.03736 | 2.15 | (0.95-4.86) | 0.06686 | 0.443964186 | 0.1 | (0.03-0.33) | 1.29E−04 | 0.040085 |
| merck_AK222828_a_at | LMAN2L | 0.11 | (0.04-0.32) | 3.89E−05 | 0.03189 | 0.24 | (0.12-0.51) | 1.91E−04 | 0.045810278 | 10.47 | (3.17-34.61) | 1.18E−04 | 0.038642 |
| merck_AL044815_at | MTMR6 | 10.63 | (3.32-34.06) | 6.99E−05 | 0.03482 | 3.77 | (1.3-10.9) | 0.01445 | 0.240575759 | 0.07 | (0.02-0.25) | 5.64E−05 | 0.033693 |
| merck_AL133569_at | LOC107987180 | 12.2 | (4.09-36.36) | 7.11E−06 | 0.02725 | 3.34 | (1.27-8.78) | 0.01443 | 0.240368307 | 0.06 | (0.02-0.2) | 6.91E−06 | 0.027253 |
| merck_AL162044_at | PCDH9 | 7.9 | (2.88-21.63) | 5.86E−05 | 0.03413 | 2.14 | (0.92-4.99) | 0.07918 | 0.472518668 | 0.11 | (0.04-0.36) | 2.35E−04 | 0.0488 |
| | PCDH9-AS4 | | | | | | | | | | | | |
| merck_AL353936_at | PSD3 | 9.11 | (3.22-25.77) | 3.12E−05 | 0.03148 | 2.61 | (1.06-6.44) | 0.03724 | 0.354817133 | 0.09 | (0.03-0.29) | 6.76E−05 | 0.034824 |
| merck_AL563761_at | — | 7.65 | (2.86-20.48) | 5.10E−05 | 0.03312 | 2.05 | (0.91-4.62) | 0.08135 | 0.476922544 | 0.1 | (0.03-0.33) | 1.07E−04 | 0.03757 |
| merck_AV762307_at | — | 0.12 | (0.04-0.33) | 5.08E−05 | 0.03312 | 0.24 | (0.11-0.5) | 1.46E−04 | 0.041503557 | 11.17 | (3.4-36.67) | 6.91E−05 | 0.034824 |
| merck_AW370282_at | — | 7.97 | (2.94-21.62) | 4.52E−05 | 0.03306 | 2.52 | (1.07-5.9) | 0.03385 | 0.341896174 | 0.08 | (0.03-0.27) | 3.39E−05 | 0.031623 |
| merck_AW665698_at | USP3-AS1 | 7.66 | (2.58-22.74) | 2.43E−04 | 0.04944 | 3.24 | (1.22-8.62) | 0.0187 | 0.269833526 | 0.08 | (0.02-0.29) | 9.86E−05 | 0.037355 |
| merck_AW771625_a_at | MAP3K6 | 0.11 | (0.04-0.29) | 1.23E−05 | 0.02735 | 0.2 | (0.09-0.44) | 4.58E−05 | 0.033063805 | 11.32 | (3.53-36.26) | 4.43E−05 | 0.033064 |
| merck_AW809224_at | CSNK1G1 | 6.41 | (2.4-17.1) | 2.05E−04 | 0.04676 | 2.3 | (1.06-5.03) | 0.03611 | 0.350871956 | 0.09 | (0.03-0.27) | 2.87E−05 | 0.030943 |
| merck_AW843987_at | GTPBP1 | 7.81 | (2.76-22.07) | 1.06E−04 | 0.03752 | 2.67 | (1.08-6.57) | 0.0329 | 0.337647514 | 0.09 | (0.03-0.31) | 9.98E−05 | 0.037355 |
| merck_AW977003_at | — | 0.17 | (0.06-0.47) | 5.70E−04 | 0.0672 | 0.29 | (0.14-0.59) | 7.39E−04 | 0.074014184 | 8.9 | (2.82-28.08) | 1.90E−04 | 0.04581 |
| merck_AY054391_at | BDNF-AS | 7.2 | (2.63-19.69) | 1.21E−04 | 0.03902 | 2.41 | (1.08-5.42) | 0.03251 | 0.335840026 | 0.08 | (0.03-0.27) | 2.60E−05 | 0.030212 |
| merck_AY147849_a_at | MIB1 | 15.51 | (4.25-56.62) | 3.32E−05 | 0.03162 | 4.89 | (1.46-16.42) | 0.01022 | 0.208143195 | 0.05 | (0.01-0.21) | 5.08E−05 | 0.033119 |
| merck_BC001905_a_at | PCSK2 | 0.13 | (0.05-0.36) | 8.91E−05 | 0.03736 | 0.25 | (0.12-0.53) | 2.67E−04 | 0.05042853 | 10.19 | (3.12-33.22) | 1.18E−04 | 0.038671 |
| merck_BC007984_at | BFSP2-AS1 | 10.67 | (3.81-29.83) | 6.43E−06 | 0.02725 | 2.39 | (1.02-5.58) | 0.04436 | 0.380430327 | 0.07 | (0.02-0.24) | 1.62E−05 | 0.027346 |
| merck_BC012447_a_at | SP110 | 0.13 | (0.05-0.34) | 5.30E−05 | 0.03312 | 0.22 | (0.1-0.46) | 8.54E−05 | 0.03697849 | 11.23 | (3.52-35.84) | 4.39E−05 | 0.033064 |
| merck_BC014119_at | LINC00824 | 0.11 | (0.04-0.32) | 6.47E−05 | 0.03482 | 0.27 | (0.13-0.56) | 4.31E−04 | 0.059118332 | 10.63 | (3.08-36.66) | 1.82E−04 | 0.044798 |
| merck_BC015877_a_at | CDH19 | 0.15 | (0.06-0.41) | 2.13E−04 | 0.04726 | 0.19 | (0.08-0.45) | 1.61E−04 | 0.042620248 | 9.98 | (3.11-32.06) | 1.11E−04 | 0.037864 |
| merck_BC019893_s_at | CD99P1 | 9.24 | (3.26-26.2) | 2.91E−05 | 0.03094 | 2.88 | (1.16-7.1) | 0.022 | 0.28730361 | 0.07 | (0.02-0.24) | 1.97E−05 | 0.028478 |
| merck_BC022892_at | IWS1 | 10.67 | (3.78-30.08) | 7.62E−06 | 0.02725 | 2.49 | (1.06-5.82) | 0.03559 | 0.348813403 | 0.07 | (0.02-0.23) | 1.20E−05 | 0.027346 |
| merck_BC034596_at | — | 6.56 | (2.31-18.6) | 4.08E−04 | 0.05788 | 3.74 | (1.49-9.36) | 0.00491 | 0.153538799 | 0.06 | (0.02-0.2) | 6.59E−06 | 0.027253 |
| merck_BC036594_at | LINCO1725 | 7.3 | (2.69-19.79) | 9.36E−05 | 0.03736 | 2.22 | (1.02-4.83) | 0.04496 | 0.382213679 | 0.08 | (0.03-0.27) | 2.73E−05 | 0.0306 |
| merck_BC039321_at | — | 10.04 | (3.67-27.46) | 6.94E−06 | 0.02725 | 1.99 | (0.85-4.65) | 0.11045 | 0.532729553 | 0.11 | (0.04-0.35) | 1.72E−04 | 0.043915 |
| merck_BC064385_at | TISP43 | 0.2 | (0.07-0.6) | 0.003781 | 0.13795 | 0.18 | (0.07-0.5) | 8.83E−04 | 0.07968467 | 12.07 | (3.38-43.14) | 1.27E−04 | 0.040085 |
| | LOC646743 | | | | | | | | | | | | |
| merck_BC110326_at | TRIQK | 11.14 | (3.07-40.48) | 2.50E−04 | 0.04966 | 4.95 | (1.48-16.52) | 0.00928 | 0.199553608 | 0.06 | (0.01-0.23) | 6.87E−05 | 0.034824 |
| merck_BC110867_s_at | CDH24 | 0.2 | (0.07-0.52) | 0.001152 | 0.08859 | 0.25 | (0.11-0.54) | 4.36E−04 | 0.059402273 | 10.18 | (3.23-32.04) | 7.32E−05 | 0.035382 |
| merck_BE219852_at | LOC101927472 | 7.16 | (2.67-19.2) | 9.28E−05 | 0.03736 | 2.14 | (0.95-4.81) | 0.06537 | 0.440652363 | 0.1 | (0.03-0.32) | 1.03E−04 | 0.037435 |
| merck_BE463518_at | LINC02004 | 10.25 | (3.68-28.57) | 8.67E−06 | 0.02735 | 2.3 | (0.99-5.37) | 0.05329 | 0.407701527 | 0.08 | (0.03-0.26) | 2.83E−05 | 0.030796 |
| merck_BE546828_at | — | 0.11 | (0.04-0.32) | 6.43E−05 | 0.03482 | 0.24 | (0.12-0.49) | 1.01E−04 | 0.037355406 | 14.01 | (4.06-48.31) | 2.94E−05 | 0.030943 |
| merck_BF342524_at | — | 0.13 | (0.04-0.36) | 9.92E−05 | 0.03736 | 0.23 | (0.11-0.5) | 1.65E−04 | 0.042993636 | 12.11 | (3.59-40.82) | 5.79E−05 | 0.034089 |
| merck_BF679703_at | — | 0.11 | (0.04-0.34) | 9.04E−05 | 0.03736 | 0.28 | (0.14-0.58) | 5.67E−04 | 0.067095592 | 10.21 | (2.97-35.11) | 2.28E−04 | 0.04813 |
| merck_BF965720_at | — | 7.12 | (2.48-20.42) | 2.61E−04 | 0.05029 | 3.57 | (1.41-9.06) | 0.00725 | 0.181228186 | 0.06 | (0.02-0.22) | 1.43E−05 | 0.027346 |
| merck_BG236239_a_at | SIRPD | 6.77 | (2.47-18.54) | 1.97E−04 | 0.04626 | 2.8 | (1.19-6.54) | 0.01788 | 0.264611925 | 0.08 | (0.02-0.26) | 2.36E−05 | 0.029051 |
| merck_BG287007_at | — | 0.15 | (0.05-0.41) | 2.27E−04 | 0.0481 | 0.26 | (0.12-0.53) | 2.76E−04 | 0.050785873 | 10.9 | (3.33-35.63) | 7.76E−05 | 0.03619 |
| merck_BG675291_at | — | 0.09 | (0.03-0.26) | 1.06E−05 | 0.02735 | 0.25 | (0.12-0.51) | 1.52E−04 | 0.041766283 | 11.28 | (3.31-38.46) | 1.08E−04 | 0.03757 |
| merck_BG740109_a_at | CLYBL | 6.43 | (2.45-16.93) | 1.62E−04 | 0.04263 | 2.03 | (0.93-4.44) | 0.07445 | 0.461049468 | 0.11 | (0.03-0.33) | 9.79E−05 | 0.037355 |
| merck_BG943385_at | THUMPD3-AS1 | 6.38 | (2.39-17.04) | 2.18E−04 | 0.04747 | 2.21 | (0.99-4.96) | 0.05307 | 0.40708177 | 0.1 | (0.03-0.32) | 9.65E−05 | 0.037355 |
| merck_BG956704_x_at | — | 4.8 | (1.79-12.91) | 0.00186 | 0.10641 | 2.9 | (1.19-7.08) | 0.01937 | 0.273642083 | 0.1 | (0.03-0.31) | 8.62E−05 | 0.037035 |
| merck_BI045425_at | — | 0.14 | (0.05-0.38) | 1.61E−04 | 0.04262 | 0.26 | (0.12-0.54) | 3.07E−04 | 0.05232964 | 10.64 | (3.21-35.28) | 1.11E−04 | 0.037864 |
| merck_BI757398_at | KLRA1P | 0.17 | (0.06-0.47) | 5.25E−04 | 0.06475 | 0.27 | (0.13-0.58) | 6.68E−04 | 0.071121654 | 8.83 | (2.76-28.31) | 2.47E−04 | 0.049436 |
| merck_BM676784_at | LINC01971 | 0.14 | (0.05-0.39) | 1.82E−04 | 0.0448 | 0.23 | (0.11-0.48) | 1.01E−04 | 0.037355406 | 14.31 | (4.23-48.44) | 1.88E−05 | 0.028478 |
| merck_BM805663_at | — | 0.12 | (0.04-0.36) | 1.48E−04 | 0.0415 | 0.21 | (0.1-0.45) | 6.17E−05 | 0.034824234 | 19.56 | (5.24-73.05) | 9.79E−06 | 0.027346 |
| merck_BQ380337_at | — | 7.7 | (2.74-21.62) | 1.08E−04 | 0.03757 | 3.21 | (1.31-7.9) | 0.01107 | 0.215503473 | 0.07 | (0.02-0.22) | 9.33E−06 | 0.027346 |
| merck_BU145850_at | SOGA1 | 0.17 | (0.06-0.45) | 3.56E−04 | 0.05498 | 0.21 | (0.09-0.48) | 2.32E−04 | 0.048677914 | 9.24 | (2.93-29.14) | 1.47E−04 | 0.041504 |
| merck_BU935068_a_at | RIMBP2 | 8.48 | (2.66-27.04) | 3.03E−04 | 0.05215 | 3.76 | (1.3-10.9) | 0.01463 | 0.241743445 | 0.08 | (0.02-0.28) | 1.06E−04 | 0.037518 |
| merck_BX095858_at | TJP2 | 4.72 | (1.74-12.77) | 0.002271 | 0.11474 | 2.77 | (1.13-6.77) | 0.02568 | 0.306276248 | 0.11 | (0.03-0.35) | 1.78E−04 | 0.044747 |
| merck_BX096093_at | NFATC3 | 9.68 | (3.25-28.83) | 4.62E−05 | 0.03306 | 3.11 | (1.18-8.2) | 0.02148 | 0.28451743 | 0.08 | (0.02-0.27) | 5.31E−05 | 0.033119 |
| | RPS12P27 | | | | | | | | | | | | |
| merck_BX097755_a_at | GDAP2 | 9.79 | (3.31-28.93) | 3.71E−05 | 0.03162 | 2.97 | (1.12-7.86) | 0.02829 | 0.318670794 | 0.09 | (0.03-0.29) | 9.06E−05 | 0.037355 |
| merck_BX102791_at | — | 6.89 | (2.6-18.28) | 1.07E−04 | 0.03757 | 2.13 | (0.95-4.8) | 0.06672 | 0.443569893 | 0.11 | (0.03-0.33) | 1.16E−04 | 0.038613 |
| merck_BX102980_at | MSN | 0.18 | (0.07-0.5) | 8.34E−04 | 0.07759 | 0.28 | (0.14-0.59) | 7.13E−04 | 0.07351598 | 9.33 | (2.94-29.55) | 1.48E−04 | 0.041504 |
| merck_BX116278_at | — | 0.07 | (0.02-0.24) | 3.67E−05 | 0.03162 | 0.3 | (0.15-0.6) | 6.16E−04 | 0.069085392 | 14.28 | (3.49-58.4) | 2.15E−04 | 0.047288 |
| merck_BX119057_at | ELFN1-AS1 | 5.46 | (2.06-14.48) | 6.52E−04 | 0.07064 | 1.91 | (0.9-4.04) | 0.09022 | 0.495013892 | 0.12 | (0.04-0.36) | 2.10E−04 | 0.047255 |
| merck_BX329942_at | PNPO | 0.05 | (0.01-0.19) | 7.70E−06 | 0.02725 | 0.28 | (0.14-0.57) | 3.75E−04 | 0.056102609 | 14.39 | (3.54-58.5) | 1.95E−04 | 0.046237 |
| merck_CA309176_at | LOC101929371 | 0.13 | (0.05-0.35) | 6.70E−05 | 0.03482 | 0.19 | (0.08-0.42) | 5.30E−05 | 0.033119082 | 12.84 | (3.97-41.57) | 2.04E−05 | 0.029019 |
| merck_CA942562_at | — | 9.61 | (3-30.72) | 1.36E−04 | 0.04048 | 3.54 | (1.21-10.31) | 0.02073 | 0.280747546 | 0.08 | (0.02-0.31) | 1.89E−04 | 0.045667 |
| merck_CD102009_at | — | 0.08 | (0.03-0.23) | 2.01E−06 | 0.02725 | 0.15 | (0.06-0.33) | 4.33E−06 | 0.027252608 | 14.84 | (4.59-47.96) | 6.56E−06 | 0.027253 |
| merck_CN388529_a_at | LAMB1 | 9.94 | (3.51-28.2) | 1.56E−05 | 0.02735 | 2.31 | (0.94-5.68) | 0.06893 | 0.448927134 | 0.11 | (0.03-0.35) | 2.04E−04 | 0.046691 |
| merck_CR742243_at | — | 6.55 | (2.4-17.92) | 2.51E−04 | 0.04966 | 2.31 | (0.98-5.41) | 0.05502 | 0.412601617 | 0.11 | (0.04-0.36) | 2.47E−04 | 0.049436 |
| merck_CR994266_a_at | JAK1 | 7.66 | (2.81-20.85) | 6.82E−05 | 0.03482 | 2.18 | (0.93-5.11) | 0.07246 | 0.456899351 | 0.11 | (0.04-0.36) | 2.16E−04 | 0.047304 |
| merck_CV392556_at | — | 17.29 | (5.93-50.43) | 1.80E−07 | 0.0209 | 2.46 | (1.05-5.75) | 0.03763 | 0.356253395 | 0.05 | (0.01-0.16) | 8.97E−07 | 0.023449 |
| merck_CX785712_at | — | 0.13 | (0.04-0.39) | 2.49E−04 | 0.04955 | 0.29 | (0.14-0.6) | 7.73E−04 | 0.075115086 | 10.04 | (2.94-34.28) | 2.31E−04 | 0.048611 |
| merck_DA214150_a_at | NDFIP1 | 6.85 | (2.58-18.2) | 1.14E−04 | 0.03815 | 2.06 | (0.91-4.64) | 0.08229 | 0.478783307 | 0.11 | (0.04-0.36) | 1.88E−04 | 0.045621 |
| merck_DA234289_at | UHRF2 | 5.26 | (1.7-16.27) | 0.003933 | 0.13976 | 3.74 | (1.29-10.82) | 0.01489 | 0.243724747 | 0.08 | (0.02-0.31) | 1.75E−04 | 0.044237 |
| merck_DB351522_at | RIN2 | 10.08 | (3.37-30.15) | 3.55E−05 | 0.03162 | 2.9 | (1.1-7.6) | 0.03097 | 0.329696004 | 0.09 | (0.03-0.3) | 1.04E−04 | 0.037518 |
| merck_DB546490_at | SLC25A48 | 12.4 | (4.35-35.32) | 2.43E−06 | 0.02725 | 1.98 | (0.85-4.62) | 0.11515 | 0.540224786 | 0.1 | (0.03-0.31) | 9.69E−05 | 0.037355 |
| merck_DN831737_at | — | 3.64 | (1.4-9.47) | 0.008089 | 0.18883 | 3.09 | (1.32-7.23) | 0.00918 | 0.198567819 | 0.09 | (0.03-0.29) | 4.18E−05 | 0.032641 |
| merck_DV080080_at | PHF2P2 | 10.02 | (3.57-28.07) | 1.18E−05 | 0.02735 | 2.19 | (1-4.76) | 0.04868 | 0.393922587 | 0.06 | (0.02-0.21) | 7.02E−06 | 0.027253 |
| merck_{—} | LRFN1 | 0.15 | (0.05-0.4) | 1.72E−04 | 0.04391 | 0.24 | (0.11-0.5) | 1.90E−04 | 0.04575582 | 10.6 | (3.31-33.99) | 7.15E−05 | 0.034986 |
| ENST00000248668_at | | | | | | | | | | | | | |
| merck_{—} | OR52B1P | 0.07 | (0.02-0.25) | 5.12E−05 | 0.03312 | 0.23 | (0.11-0.47) | 5.69E−05 | 0.033692775 | 22.93 | (5.53-95.12) | 1.59E−05 | 0.027346 |
| ENST00000316506_a_at | | | | | | | | | | | | | |
| merck_{—} | BCAS4 | 0.12 | (0.04-0.31) | 1.54E−05 | 0.02735 | 0.21 | (0.1-0.47) | 9.74E−05 | 0.037355406 | 8.6 | (2.79-26.52) | 1.81E−04 | 0.044798 |
| ENST00000323496_x_at | | | | | | | | | | | | | |
| merck_{—} | LOC107985946 | 0.13 | (0.05-0.35) | 6.16E−05 | 0.03482 | 0.23 | (0.11-0.48) | 1.15E−04 | 0.038254005 | 10.6 | (3.32-33.83) | 6.66E−05 | 0.034824 |
| ENST00000327196_at | | | | | | | | | | | | | |
| merck_{—} | RPS17P5 | 6.82 | (2.42-19.17) | 2.76E−04 | 0.05079 | 2.6 | (1.06-6.41) | 0.03766 | 0.356309272 | 0.11 | (0.03-0.35) | 2.18E−04 | 0.047468 |
| ENST00000331146_at | | | | | | | | | | | | | |
| merck_{—} | TAF4B | 8.27 | (3.06-22.33) | 3.10E−05 | 0.03148 | 2.17 | (0.93-5.06) | 0.07324 | 0.458339281 | 0.11 | (0.03-0.34) | 1.37E−04 | 0.040494 |
| ENST00000341244_a_at | | | | | | | | | | | | | |
| merck_{—} | FAM92A | 0.09 | (0.03-0.3) | 5.40E−05 | 0.03318 | 0.11 | (0.04-0.32) | 5.34E−05 | 0.033119082 | 12.13 | (3.34-43.99) | 1.47E−04 | 0.041504 |
| ENST00000359421_at | | | | | | | | | | | | | |
| merck_{—} | OR14A2 | 19.28 | (6.14-60.48) | 3.94E−07 | 0.02345 | 1.43 | (0.7-2.95) | 0.32532 | 0.759033056 | 0.05 | (0.01-0.17) | 2.91E−06 | 0.027253 |
| ENST00000366485_at | | | | | | | | | | | | | |
| merck_{—} | SHISA7 | 0.13 | (0.04-0.36) | 9.42E−05 | 0.03736 | 0.24 | (0.11-0.51) | 1.87E−04 | 0.045613476 | 11.47 | (3.44-38.28) | 7.22E−05 | 0.034986 |
| ENST00000376325_at | | | | | | | | | | | | | |
| merck_{—} | REXO1L2P | 6.15 | (2.3-16.47) | 3.00E−04 | 0.05212 | 2.12 | (0.95-4.74) | 0.06817 | 0.447120854 | 0.12 | (0.04-0.36) | 2.05E−04 | 0.046764 |
| ENST00000378999_s_at | | | | | | | | | | | | | |
| merck_{—} | MCHR1 | 6.29 | (2.38-16.6) | 2.05E−04 | 0.04674 | 1.77 | (0.83-3.75) | 0.13924 | 0.576414125 | 0.12 | (0.04-0.37) | 2.43E−04 | 0.049436 |
| ENST00000381435_at | | | | | | | | | | | | | |
| merck_G65625_at | — | 11.09 | (3.46-35.52) | 5.08E−05 | 0.03312 | 3.81 | (1.31-11.07) | 0.01387 | 0.236245861 | 0.07 | (0.02-0.24) | 4.46E−05 | 0.033064 |
| merck_NC_001405_ORF_1045_s_at | — | 0.09 | (0.03-0.27) | 1.84E−05 | 0.02848 | 0.22 | (0.11-0.46) | 5.12E−05 | 0.033119082 | 14.56 | (4.24-50.04) | 2.11E−05 | 0.029039 |
| merck_NM_000258_at | MYL3 | 9.46 | (3.38-26.47) | 1.85E−05 | 0.02848 | 1.98 | (0.91-4.29) | 0.08476 | 0.484222405 | 0.08 | (0.02-0.26) | 2.71E−05 | 0.0306 |
| merck_NM_000273_at | GPR143 | 0.13 | (0.04-0.38) | 2.19E−04 | 0.04753 | 0.28 | (0.14-0.59) | 6.60E−04 | 0.070759139 | 10.6 | (3.06-36.73) | 1.96E−04 | 0.04626 |
| merck_NM_000782_at | CYP24A1 | 0.09 | (0.03-0.27) | 2.00E−05 | 0.02857 | 0.23 | (0.11-0.49) | 1.14E−04 | 0.038189964 | 12.81 | (3.7-44.31) | 5.67E−05 | 0.033693 |
| merck_NM_000873_at | ICAM2 | 0.16 | (0.06-0.44) | 4.41E−04 | 0.05971 | 0.28 | (0.14-0.59) | 7.37E−04 | 0.074014184 | 9.22 | (2.83-30.01) | 2.24E−04 | 0.048058 |
| merck_NM_001001706_at | AK9 | 7.38 | (2.48-21.95) | 3.23E−04 | 0.0534 | 3.04 | (1.14-8.05) | 0.02563 | 0.306089358 | 0.1 | (0.03-0.33) | 2.12E−04 | 0.047264 |
| merck_NM_001002247_at | ANAPC11 | 0.08 | (0.02-0.29) | 1.35E−04 | 0.04037 | 0.29 | (0.14-0.58) | 5.24E−04 | 0.06475289 | 16.36 | (3.93-68.13) | 1.24E−04 | 0.039414 |
| merck_NM_001004434_s_at | SLC30A2 | 0.07 | (0.02-0.22) | 7.31E−06 | 0.02725 | 0.26 | (0.13-0.54) | 2.57E−04 | 0.050108064 | 12.24 | (3.33-45.04) | 1.64E−04 | 0.042833 |
| merck_NM_001004752_at | OR51F1 | 10.18 | (3.17-32.67) | 9.62E−05 | 0.03736 | 3.57 | (1.23-10.33) | 0.01912 | 0.271848701 | 0.08 | (0.02-0.29) | 1.27E−04 | 0.040085 |
| merck_NM_001012505_a_at | FOXP1 | 8.53 | (3.02-24.09) | 5.23E−05 | 0.03312 | 3.41 | (1.37-8.5) | 0.00861 | 0.1934917 | 0.06 | (0.02-0.2) | 5.38E−06 | 0.027253 |
| merck_NM_001024599_s_at | HIST2H2BF | 8.88 | (3.23-24.37) | 2.25E−05 | 0.02905 | 2.48 | (1.06-5.81) | 0.03723 | 0.354799945 | 0.08 | (0.02-0.26) | 2.68E−05 | 0.030477 |
| merck_NM_001039753_s_at | EML6 | 0.12 | (0.04-0.33) | 5.82E−05 | 0.03409 | 0.13 | (0.05-0.33) | 1.66E−05 | 0.027345536 | 17.21 | (5.08-58.38) | 4.94E−06 | 0.027253 |
| merck_NM_001039_at | SCNN1G | 0.13 | (0.05-0.35) | 5.37E−05 | 0.03312 | 0.17 | (0.08-0.4) | 3.90E−05 | 0.031886786 | 12.72 | (3.93-41.12) | 2.18E−05 | 0.029051 |
| merck_NM_001294_at | CLPTM1 | 0.1 | (0.03-0.29) | 2.78E−05 | 0.0308 | 0.26 | (0.13-0.53) | 2.12E−04 | 0.047263991 | 11.07 | (3.27-37.52) | 1.13E−04 | 0.038005 |
| merck_NM_001431_s_at | EPB41L2 | 6.9 | (2.47-19.29) | 2.34E−04 | 0.04878 | 2.64 | (1.07-6.5) | 0.03456 | 0.344844557 | 0.1 | (0.03-0.33) | 1.53E−04 | 0.041846 |
| merck_NM001494_at | GDI2 | 7.32 | (2.75-19.49) | 6.86E−05 | 0.03482 | 2.14 | (0.95-4.83) | 0.06614 | 0.442547335 | 0.1 | (0.03-0.32) | 8.43E−05 | 0.036978 |
| merck_NM_001883_at | CRHR2 | 9.12 | (3.1-26.88) | 6.09E−05 | 0.03482 | 3.63 | (1.38-9.55) | 0.00918 | 0.198588716 | 0.06 | (0.02-0.21) | 8.09E−06 | 0.027253 |
| merck_NM_001993_at | F3 | 12.06 | (4.3-33.83) | 2.25E−06 | 0.02725 | 1.33 | (0.64-2.76) | 0.44992 | 0.832172801 | 0.1 | (0.03-0.32) | 1.01E−04 | 0.037355 |
| merck_NM_002501_s_at | NFIX | 0.13 | (0.05-0.37) | 9.05E−05 | 0.03736 | 0.2 | (0.09-0.44) | 4.77E−05 | 0.033119082 | 14.15 | (4.31-46.46) | 1.25E−05 | 0.027346 |
| merck_NM_003920_s_at | TIMELESS | 0.12 | (0.04-0.35) | 7.90E−05 | 0.03655 | 0.26 | (0.13-0.55) | 3.81E−04 | 0.056284608 | 9.25 | (2.84-30.13) | 2.22E−04 | 0.047784 |
| merck_NM_003954_at | MAP3K14 | 0.12 | (0.05-0.34) | 4.60E−05 | 0.03306 | 0.22 | (0.1-0.48) | 1.07E−04 | 0.037570373 | 10.58 | (3.31-33.86) | 6.98E−05 | 0.034824 |
| merck_NM_004318_at | ASPH | 6.75 | (2.41-18.9) | 2.76E−04 | 0.05083 | 2.87 | (1.16-7.12) | 0.02268 | 0.291108094 | 0.09 | (0.03-0.3) | 8.16E−05 | 0.036737 |
| merck_NM_004387_at | NKX2-5 | 0.11 | (0.04-0.33) | 6.70E−05 | 0.03482 | 0.27 | (0.13-0.56) | 3.55E−04 | 0.054982332 | 10.66 | (3.14-36.18) | 1.46E−04 | 0.041504 |
| merck_NM_004422_at | DVL2 | 0.16 | (0.06-0.43) | 3.09E−04 | 0.05243 | 0.2 | (0.08-0.47) | 2.08E−04 | 0.04703142 | 9.85 | (3.07-31.64) | 1.21E−04 | 0.039022 |
| merck_NM_004683_s_at | RGN | 0.1 | (0.04-0.29) | 1.86E−05 | 0.02848 | 0.14 | (0.06-0.37) | 4.61E−05 | 0.033063805 | 9.95 | (3.02-32.85) | 1.62E−04 | 0.042632 |
| merck_NM_005184_s_at | CALM3 | 0.08 | (0.02-0.24) | 1.44E−05 | 0.02735 | 0.26 | (0.13-0.53) | 2.13E−04 | 0.047263991 | 13.26 | (3.61-48.7) | 9.78E−05 | 0.037355 |
| merck_NM_006143_at | GPR19 | 0.13 | (0.05-0.34) | 3.72E−05 | 0.03162 | 0.22 | (0.1-0.48) | 1.48E−04 | 0.041538264 | 8.57 | (2.76-26.62) | 2.01E−04 | 0.046463 |
| merck_NM_006268_at | DPF2 | 0.16 | (0.06-0.43) | 2.66E−04 | 0.05041 | 0.23 | (0.11-0.51) | 2.66E−04 | 0.050411995 | 9.49 | (3.01-29.94) | 1.23E−04 | 0.039214 |
| merck_NM_007179_at | INSL6 | 16.37 | (4.45-60.29) | 2.63E−05 | 0.03031 | 5.86 | (1.74-19.78) | 0.00438 | 0.146142686 | 0.03 | (0.01-0.15) | 6.21E−06 | 0.027253 |
| merck_NM_014184_s_at | CNIH4 | 0.17 | (0.06-0.44) | 3.01E−04 | 0.05212 | 0.21 | (0.1-0.47) | 1.51E−04 | 0.041766283 | 10.38 | (3.34-32.3) | 5.36E−05 | 0.033119 |
| merck_NM014230_s_at | SRP68 | 0.21 | (0.08-0.58) | 0.002323 | 0.11599 | 0.29 | (0.14-0.61) | 0.00108 | 0.086110602 | 9.25 | (2.89-29.64) | 1.81E−04 | 0.044798 |
| merck_NM_014553_at | TFCP2L1 | 0.18 | (0.07-0.49) | 7.24E−04 | 0.07378 | 0.24 | (0.11-0.52) | 3.53E−04 | 0.054933977 | 10.63 | (3.25-34.7) | 9.06E−05 | 0.037355 |
| merck_NM_015058_at | VWA8 | 6.11 | (2.16-17.31) | 6.55E−04 | 0.07066 | 3.39 | (1.3-8.86) | 0.01256 | 0.227192335 | 0.08 | (0.02-0.27) | 3.58E−05 | 0.031623 |
| merck_NM_015832_a_at | MBD2 | 8.52 | (3.12-23.31) | 2.99E−05 | 0.03094 | 1.84 | (0.87-3.91) | 0.1119 | 0.534970919 | 0.09 | (0.03-0.28) | 3.75E−05 | 0.031623 |
| merck_NM_016593_at | CYP39A1 | 6.51 | (2.42-17.53) | 2.12E−04 | 0.04726 | 2.39 | (1.02-5.57) | 0.04379 | 0.378543596 | 0.11 | (0.03-0.33) | 1.15E−04 | 0.038254 |
| merck_NM_017916_at | PIH1D1 | 0.08 | (0.03-0.25) | 5.65E−06 | 0.02725 | 0.17 | (0.08-0.37) | 9.23E−06 | 0.027345536 | 17.69 | (5.21-60.1) | 4.14E−06 | 0.027253 |
| merck_NM_018055_at | NODAL | 0.11 | (0.04-0.32) | 3.93E−05 | 0.03189 | 0.23 | (0.11-0.48) | 1.01E−04 | 0.037355406 | 11.97 | (3.6-39.84) | 5.21E−05 | 0.033119 |
| merck_NM_018219_at | CCDC87 | 8.2 | (2.91-23.08) | 6.84E−05 | 0.03482 | 2.5 | (1.02-6.13) | 0.04531 | 0.383198098 | 0.1 | (0.03-0.33) | 1.56E−04 | 0.042146 |
| merck_NM_020457_at | THAP11 | 0.12 | (0.04-0.33) | 3.50E−05 | 0.03162 | 0.24 | (0.11-0.51) | 1.88E−04 | 0.045667188 | 8.83 | (2.81-27.71) | 1.89E−04 | 0.045707 |
| merck_NM_020651_at | PELI1 | 8.02 | (2.7-23.85) | 1.82E−04 | 0.0448 | 3.16 | (1.19-8.35) | 0.02052 | 0.279698435 | 0.08 | (0.02-0.29) | 1.02E−04 | 0.037355 |
| merck_NM_020677_s_at | NMRAL1 | 0.09 | (0.03-0.27) | 1.89E−05 | 0.02848 | 0.19 | (0.09-0.41) | 1.93E−05 | 0.028478422 | 19.1 | (5.34-68.26) | 5.66E−06 | 0.027253 |
| merck_NM_020774_at | MIB1 | 12.73 | (3.94-41.14) | 2.12E−05 | 0.02904 | 3.76 | (1.3-10.86) | 0.01455 | 0.24122981 | 0.06 | (0.02-0.23) | 3.50E−05 | 0.031623 |
| merck_NM_021912_at | GABRB3 | 0.08 | (0.03-0.23) | 2.44E−06 | 0.02725 | 0.16 | (0.07-0.36) | 1.66E−05 | 0.027345536 | 12.24 | (3.72-40.24) | 3.72E−05 | 0.031623 |
| merck_NM_021946_s_at | BCORL1 | 0.1 | (0.03-0.27) | 7.90E−06 | 0.02725 | 0.18 | (0.08-0.4) | 2.22E−05 | 0.029051464 | 12.88 | (3.97-41.78) | 2.07E−05 | 0.029039 |
| merck_NM_021983_x_at | HLA-DRB4 | 0.09 | (0.03-0.28) | 1.94E−05 | 0.02848 | 0.27 | (0.13-0.56) | 3.55E−04 | 0.054982332 | 9.81 | (2.9-33.2) | 2.40E−04 | 0.049331 |
| merck_NM_022358_at | KCNK15 | 0.11 | (0.04-0.33) | 7.86E−05 | 0.03646 | 0.28 | (0.14-0.57) | 4.46E−04 | 0.059751206 | 10.28 | (3.02-34.97) | 1.90E−04 | 0.045756 |
| merck_NM_025214_at | CCDC68 | 5.95 | (2.19-16.18) | 4.78E−04 | 0.06192 | 2.65 | (1.13-6.24) | 0.02569 | 0.306276248 | 0.09 | (0.03-0.3) | 8.00E−05 | 0.036707 |
| merck_NM_030944_at | LINC00597 | 9.12 | (3.23-25.74) | 2.98E−05 | 0.03094 | 2.46 | (1-6.07) | 0.05099 | 0.400428172 | 0.1 | (0.03-0.33) | 1.37E−04 | 0.040515 |
| merck_NM_032143_a_at | ZRANB3 | 9.37 | (3.39-25.91) | 1.64E−05 | 0.02735 | 1.83 | (0.84-4) | 0.1281 | 0.559452987 | 0.09 | (0.03-0.3) | 7.53E−05 | 0.035643 |
| merck_NM_032594_at | INSM2 | 5.96 | (2.2-16.13) | 4.37E−04 | 0.05942 | 2.5 | (1.06-5.86) | 0.03572 | 0.349233007 | 0.1 | (0.03-0.33) | 1.32E−04 | 0.040145 |
| merck_NM_052910_at | SLITRK1 | 10.91 | (2.99-39.76) | 2.93E−04 | 0.05172 | 4.5 | (1.34-15.11) | 0.01487 | 0.243608058 | 0.07 | (0.02-0.29) | 2.55E−04 | 0.049968 |
| merck_NM_058165_at | MOGAT1 | 7.6 | (2.57-22.45) | 2.45E−04 | 0.04944 | 3.14 | (1.18-8.34) | 0.02139 | 0.284138026 | 0.09 | (0.03-0.3) | 1.18E−04 | 0.038642 |
| merck_NM_058219_at | EXOSC6 | 0.13 | (0.05-0.36) | 6.78E−05 | 0.03482 | 0.24 | (0.11-0.51) | 2.25E−04 | 0.048058073 | 9.16 | (2.9-28.93) | 1.60E−04 | 0.04262 |
| merck_NM_133262_at | ATP6V1G3 | 7.71 | (2.6-22.9) | 2.34E−04 | 0.04878 | 2.94 | (1.11-7.77) | 0.02956 | 0.324027699 | 0.1 | (0.03-0.34) | 2.47E−04 | 0.049436 |
| merck_NM_133492_at | ACER1 | 0.14 | (0.05-0.38) | 8.86E−05 | 0.03736 | 0.22 | (0.1-0.47) | 1.05E−04 | 0.037518224 | 9.88 | (3.21-30.45) | 6.59E−05 | 0.034824 |
| merck_NM_138447_a_at | ZNF689 | 0.15 | (0.06-0.4) | 1.33E−04 | 0.04015 | 0.21 | (0.09-0.48) | 1.76E−04 | 0.044352246 | 9.39 | (2.93-30.07) | 1.64E−04 | 0.042833 |
| merck_NM_139174_at | ADAD2 | 0.09 | (0.03-0.29) | 5.76E−05 | 0.03399 | 0.24 | (0.12-0.5) | 1.06E−04 | 0.037518224 | 16.66 | (4.47-62.13) | 2.82E−05 | 0.030796 |
| merck_NM_139315_s_at | TAF6 | 0.14 | (0.05-0.39) | 1.19E−04 | 0.03867 | 0.21 | (0.1-0.48) | 2.00E−04 | 0.04637483 | 8.72 | (2.78-27.38) | 2.07E−04 | 0.046967 |
| merck_NM_145270_at | PRR35 | 0.12 | (0.04-0.34) | 5.07E−05 | 0.03312 | 0.2 | (0.09-0.43) | 4.79E−05 | 0.033119082 | 13.61 | (4.12-44.95) | 1.83E−05 | 0.028478 |
| merck_NM_152730_s_at | TBC1D32 | 15.24 | (5.24-44.29) | 5.60E−07 | 0.02345 | 2.18 | (0.94-5.07) | 0.0704 | 0.45214295 | 0.07 | (0.02-0.22) | 1.04E−05 | 0.027346 |
| merck_NM_153274_at | BEST4 | 0.1 | (0.04-0.3) | 3.65E−05 | 0.03162 | 0.12 | (0.05-0.32) | 1.94E−05 | 0.028478422 | 16.05 | (4.65-55.4) | 1.12E−05 | 0.027346 |
| merck_NM_173798_at | ZCCHC12 | 0.08 | (0.03-0.24) | 2.36E−06 | 0.02725 | 0.17 | (0.08-0.37) | 8.34E−06 | 0.027345536 | 14.15 | (4.36-45.92) | 1.02E−05 | 0.027346 |
| merck_NM_174962_x_at | SSX9 | 0.08 | (0.02-0.28) | 1.02E−04 | 0.03736 | 0.3 | (0.15-0.61) | 8.34E−04 | 0.077590388 | 14.35 | (3.5-58.8) | 2.14E−04 | 0.047288 |
| merck_NM_176889_at | TAS2R20 | 9.8 | (3.32-28.97) | 3.64E−05 | 0.03162 | 2.97 | (1.13-7.83) | 0.02728 | 0.314679432 | 0.08 | (0.02-0.29) | 8.40E−05 | 0.036978 |
| merck_NM_178012_at | TUBB2B | 0.2 | (0.08-0.55) | 0.001562 | 0.09975 | 0.22 | (0.09-0.51) | 4.04E−04 | 0.057704837 | 10.97 | (3.37-35.75) | 7.05E−05 | 0.034828 |
| merck_NM_181608_x_at | KRTAP19-2 | 0.09 | (0.03-0.27) | 1.52E−05 | 0.02735 | 0.24 | (0.11-0.49) | 9.96E−05 | 0.037355406 | 12.58 | (3.67-43.14) | 5.62E−05 | 0.033693 |
| merck_NM_182505_at | C9orf85 | 9.07 | (3.19-25.81) | 3.57E−05 | 0.03162 | 3 | (1.22-7.38) | 0.01689 | 0.257661496 | 0.07 | (0.02-0.23) | 1.33E−05 | 0.027346 |
| merck_NM_182800_at | SRRT | 0.09 | (0.03-0.3) | 6.40E−05 | 0.03482 | 0.29 | (0.14-0.59) | 6.57E−04 | 0.070660281 | 11.41 | (3.13-41.61) | 2.27E−04 | 0.048105 |
| merck_NM_182905_s_at | WASHC1 | 0.13 | (0.05-0.34) | 5.14E−05 | 0.03312 | 0.23 | (0.11-0.5) | 1.62E−04 | 0.042632345 | 9.75 | (3.03-31.35) | 1.32E−04 | 0.040145 |
| merck_NM_198994_at | TGM6 | 0.08 | (0.03-0.21) | 1.24E−06 | 0.02504 | 0.17 | (0.07-0.37) | 1.30E−05 | 0.027345536 | 12.72 | (3.89-41.56) | 2.57E−05 | 0.030212 |
| merck_NM_199180_at | KIRREL2 | 0.14 | (0.05-0.39) | 1.51E−04 | 0.04177 | 0.24 | (0.11-0.51) | 2.25E−04 | 0.048058073 | 10 | (3.14-31.86) | 9.92E−05 | 0.037355 |
| merck_VZV_OKA_ORF41_s_at | — | 0.12 | (0.04-0.33) | 3.92E−05 | 0.03189 | 0.14 | (0.06-0.35) | 2.33E−05 | 0.029051464 | 12.64 | (3.9-40.99) | 2.38E−05 | 0.029051 |
| merck_VZV_OKA_ORF9_s_at | — | 0.13 | (0.05-0.35) | 5.95E−05 | 0.03444 | 0.24 | (0.11-0.51) | 2.34E−04 | 0.048800286 | 9.15 | (2.87-29.22) | 1.84E−04 | 0.04516 |
| merck_X85629_at | — | 11.02 | (3.98-30.55) | 3.95E−06 | 0.02725 | 2.66 | (1.18-6) | 0.01867 | 0.269833526 | 0.05 | (0.02-0.17) | 1.03E−06 | 0.023449 |
| merck_X98206_at | ARID1B | 8.73 | (3.07-24.78) | 4.73E−05 | 0.03312 | 2.84 | (1.15-7) | 0.02339 | 0.294836152 | 0.08 | (0.02-0.26) | 3.13E−05 | 0.031476 |
| | MIR4466 | | | | | | | | | | | | |
| merck_XM_063314_at | OR11H5P | 10.56 | (2.88-38.76) | 3.81E−04 | 0.05628 | 5.35 | (1.59-18.01) | 0.00681 | 0.175792963 | 0.05 | (0.01-0.21) | 4.70E−05 | 0.033119 |
| merck_XM_210303_at | — | 0.12 | (0.04-0.37) | 2.92E−04 | 0.05172 | 0.29 | (0.14-0.59) | 6.08E−04 | 0.068789404 | 12.85 | (3.49-47.37) | 1.25E−04 | 0.039651 |
| merck_XM_373233_x_at | — | 7.64 | (2.69-21.66) | 1.32E−04 | 0.04015 | 2.5 | (1.02-6.12) | 0.04572 | 0.384379613 | 0.11 | (0.03-0.35) | 1.96E−04 | 0.04626 |
| merck_XM_378090_at | — | 0.1 | (0.04-0.28) | 1.01E−05 | 0.02735 | 0.2 | (0.09-0.45) | 9.29E−05 | 0.037355406 | 8.99 | (2.81-28.8) | 2.16E−04 | 0.047312 |
| merck_XM_926011_at | LOC642515 | 0.07 | (0.02-0.2) | 9.27E−07 | 0.02345 | 0.18 | (0.08-0.4) | 3.19E−05 | 0.031578951 | 10.55 | (3.24-34.36) | 9.27E−05 | 0.037355 |
| merck_XM_928173_s_at | PPIAL4A | 4.9 | (1.85-12.99) | 0.001383 | 0.09443 | 2.13 | (0.98-4.66) | 0.05729 | 0.418885847 | 0.12 | (0.04-0.37) | 2.52E−04 | 0.049663 |
| | PPIAL4C | | | | | | | | | | | | |
| | PPIAL4D | | | | | | | | | | | | |
| | PPIAL4E | | | | | | | | | | | | |
| | PPIAL4F | | | | | | | | | | | | |
| | PPIAL4G | | | | | | | | | | | | |
| merck_XM_928586_x_at | FAM207BP | 0.21 | (0.08-0.55) | 0.001728 | 0.10319 | 0.27 | (0.13-0.56) | 4.31E−04 | 0.059118332 | 11.73 | (3.67-37.53) | 3.32E−05 | 0.031623 |
| | FAM207CP | | | | | | | | | | | | |
| merck_XM_929767_at | — | 0.1 | (0.03-0.3) | 7.15E−05 | 0.03499 | 0.29 | (0.15-0.59) | 6.42E−04 | 0.070111466 | 11.47 | (3.15-41.71) | 2.13E−04 | 0.047264 |
| merck_XM_933463_at | — | 0.14 | (0.05-0.38) | 1.15E−04 | 0.03825 | 0.25 | (0.12-0.52) | 2.52E−04 | 0.049663475 | 9.33 | (2.95-29.44) | 1.40E−04 | 0.040803 |
| merck_XM_937104_at | — | 0.17 | (0.06-0.46) | 4.63E−04 | 0.0608 | 0.26 | (0.13-0.55) | 4.31E−04 | 0.059118332 | 9.53 | (3-30.27) | 1.31E−04 | 0.040145 |
| merck_XM_940419_at | SLC9B1P1 | 0.12 | (0.04-0.36) | 1.29E−04 | 0.04009 | 0.26 | (0.13-0.55) | 3.15E−04 | 0.052817414 | 11.61 | (3.4-39.68) | 9.21E−05 | 0.037355 |
| merck_XM_943662_at | — | 0.12 | (0.04-0.35) | 1.21E−04 | 0.03902 | 0.29 | (0.14-0.59) | 6.59E−04 | 0.070660281 | 10 | (2.91-34.3) | 2.51E−04 | 0.049663 |
| merck_XM_945363_at | — | 7.59 | (2.78-20.73) | 7.66E−05 | 0.03597 | 1.99 | (0.89-4.46) | 0.09449 | 0.503642535 | 0.11 | (0.04-0.35) | 1.84E−04 | 0.04516 |
| merck_hCT1651346_at | RPS28P1 | 9.22 | (3.09-27.48) | 6.71E−05 | 0.03482 | 2.87 | (1.09-7.56) | 0.03333 | 0.339626483 | 0.09 | (0.03-0.32) | 1.69E−04 | 0.043573 |
| merck_hCT1651838_3_at | LOC100288437 | 13.07 | (4.6-37.16) | 1.43E−06 | 0.026 | 2.82 | (1.25-6.37) | 0.01259 | 0.227358462 | 0.04 | (0.01-0.13) | 2.30E−07 | 0.020898 |
| merck_hCT1812405_2_at | ELOCP31 | 9.65 | (3.39-27.49) | 2.19E−05 | 0.02905 | 2.87 | (1.16-7.09) | 0.02269 | 0.291120926 | 0.07 | (0.02-0.24) | 1.97E−05 | 0.028478 |
| merck_hCT1846664_1_at | — | 9.32 | (3.42-25.39) | 1.27E−05 | 0.02735 | 1.85 | (0.85-4.01) | 0.1215 | 0.549829621 | 0.09 | (0.03-0.29) | 5.22E−05 | 0.033119 |
| merck_hCT2307348_at | — | 0.07 | (0.02-0.26) | 6.24E−05 | 0.03482 | 0.3 | (0.15-0.61) | 8.11E−04 | 0.076586776 | 14.49 | (3.5-59.98) | 2.26E−04 | 0.048104 |
| merck_hsa_mir_153_1_x_at | MIR153-1 | 0.13 | (0.04-0.38) | 2.24E−04 | 0.04806 | 0.27 | (0.13-0.56) | 4.46E−04 | 0.059751206 | 11.51 | (3.32-39.94) | 1.19E−04 | 0.038671 |

### (b) Pubmed Analysis of the Gene Signature (279 Probe Sets)

The 279 probe sets were linked to 198 genes. Among them, there were 76 genes were associated with colon with 22 linked to colon cancer. These colon cancer associated genes were AGR2, BEST4, CRHR2, CRK, CYP24A1, DVL2, ELFN1-AS1, F3, FIP1L1, FOXP1, ICAM2, JAK1, MAP3K14, MBD2, MIB1, MSN, NKX2-5, NODAL, THUMPD3-AS1, TIMELESS, TJP2, USP46.

### (3) PCA Model

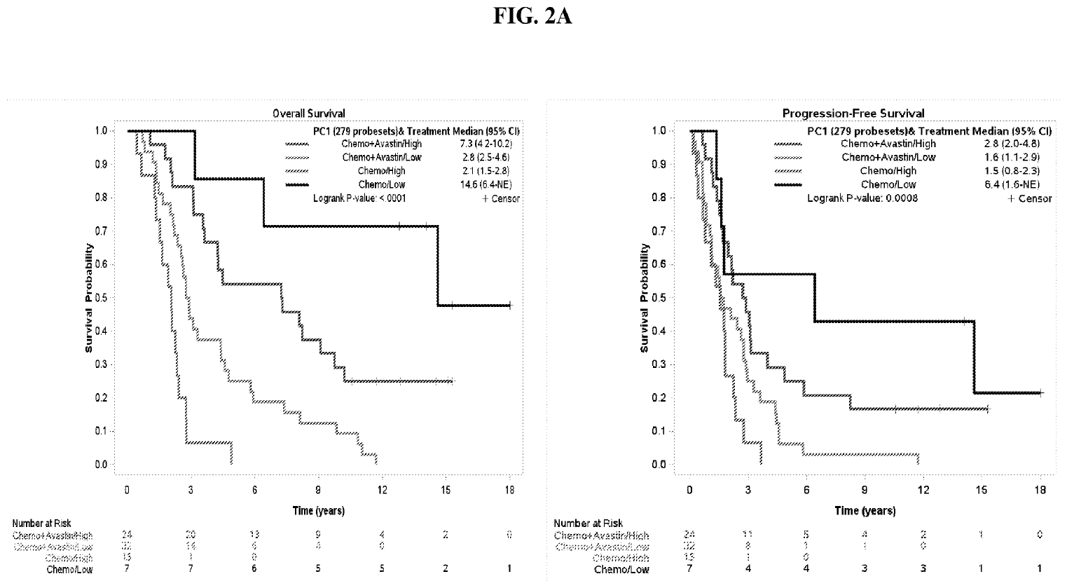

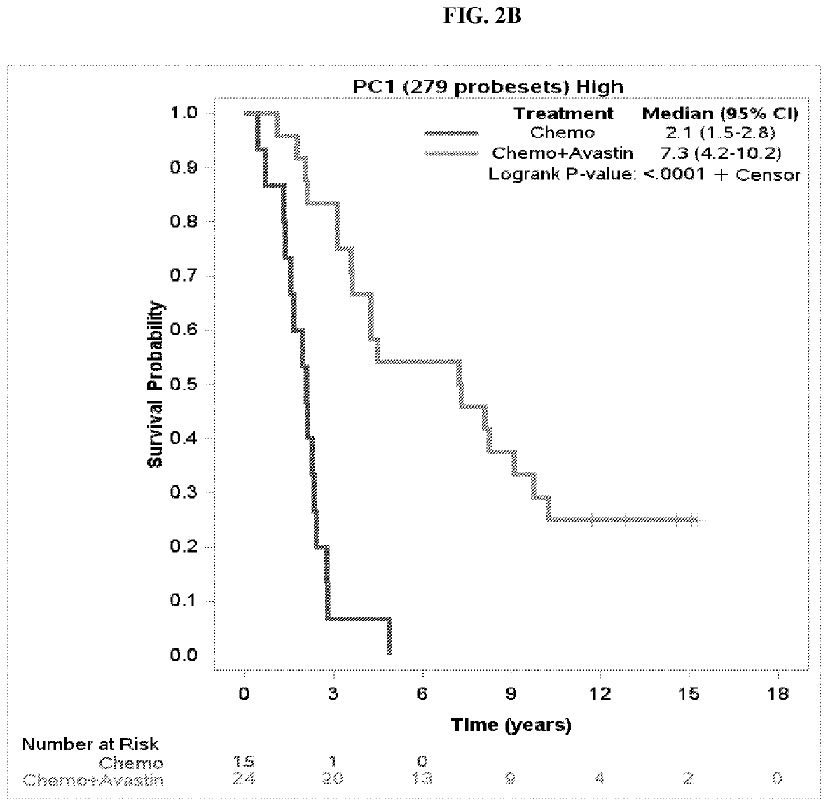

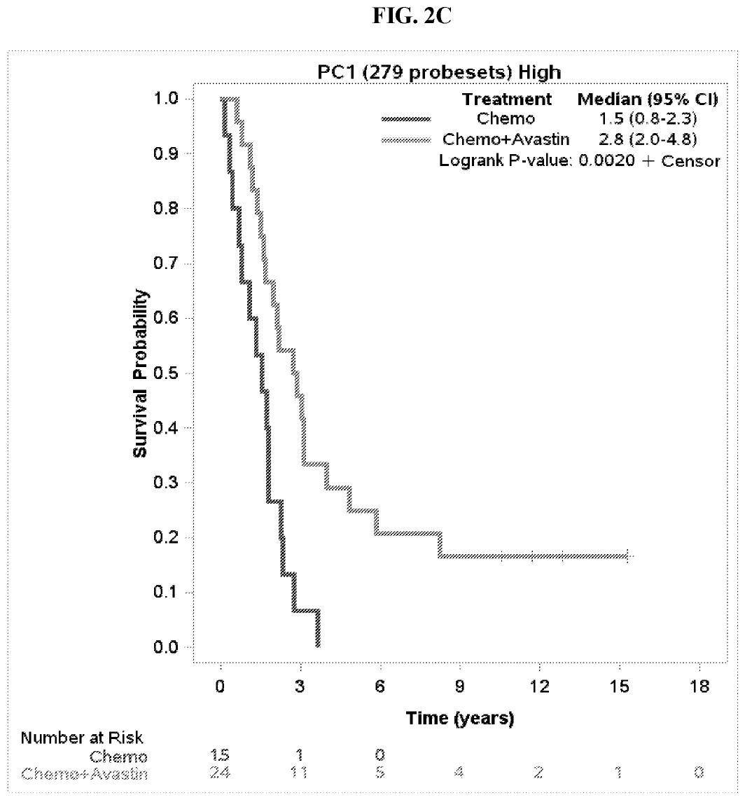

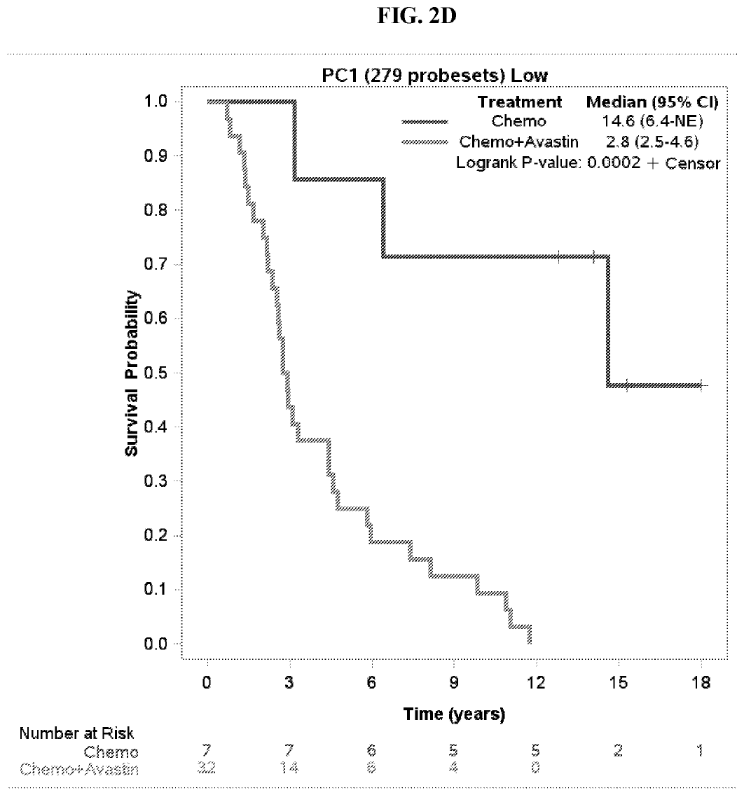

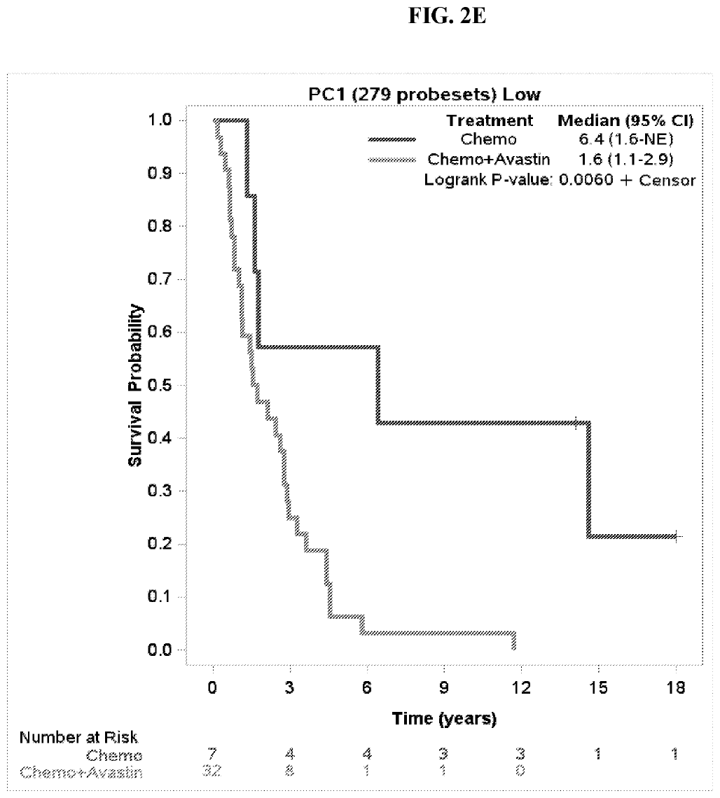

PCA was uses to summarize the 279 significant probe sets and showed efficiency of data reduction with the first principal component (PC1) explained 14% 20% total variation, higher than 11% by the use of whole genes. The median cutoff PC1 was incorporated in the interaction model with the treatment variable to build a predictive model in estimating treatment effect. Analysis result showed the interaction model was able to predict an AVASTIN® effect and also a chemotherapy effect in terms of OS (p<0.0001; FIG. 2A and Table 2). Specifically, patients treated with AVASTIN® tend to improve in survival if the PC1 score was high (median OS: 7.27 years in AVASTIN®+chemotherapy versus 2.08 years in chemotherapy only; p<0.0001; FIG. 2B, and Table 3). On the other hand, a patient with a low PC1 score seems to have better OS if treated with chemotherapy only (median OS: not reached in chemotherapy only versus 2.91 years in AVASTIN®+chemotherapy; p=2e-04; FIG. 2C and Table 4). Patients received chemotherapy only had worse overall survival if the PC1 score from 279 significant probe sets is high (hazard ratio 18.28, 95% CI 4.93-67.73); patients with low PC1 also had worse overall survival if they treated with AVASTIN® (hazard ratio 7.11, 95% CI 2.1-24.08); PC1 score from 279 significant probe sets and treatment type interacts because the relationship between treatment type and overall survival changes depending on PC1 score.

TABLE 2
Median Survival Time of Interaction Model in Moffitt Cohort

| strata | median | lower | upper |
|---|---|---|---|
| g1 = Chemo + | 7.269863 | 4.246575 | 10.230137 |
| AVASTIN ®_High_PC1 | | | |
| g1 = Chemo + | 2.913699 | 2.597260 | 4.750685 |
| AVASTIN ®_Low_PC1 | | | |
| g1 = Chemo_High_PC1 | 2.079452 | 1.542466 | 2.780822 |
| g1 = Chemo_Low_PC1 | NA | 6.416438 | NA |

### Interaction Model: High PC1

TABLE 3
Median Survival Time of High PC1 in Moffitt Cohort

| strata | median | lower | upper |
|---|---|---|---|
| Treatment = Chemo | 2.079452 | 1.542466 | 2.780822 |
| Treatment = Chemo + AVASTIN ® | 7.269863 | 4.246575 | 10.230137 |

### Interaction Model: Low PC1

TABLE 4
Median Survival Time of Low PC1 in Moffitt Cohort

| strata | median | lower | upper |
|---|---|---|---|
| Treatment = Chemo | NA | 6.416438 | NA |
| Treatment = Chemo + AVASTIN ® | 2.913699 | 2.597260 | 4.750685 |

### (4) Validation in Moffitt Consortium Cohort

Data were collected from 39 mCRC patients in the Moffitt Consortium cohort. Overall survival (OS) was the primary endpoint (n=39 death event; 67%). Nine patients (23%) received Avastin and chemotherapy while 30 patients (77%) had chemotherapy only. Gene expression data were generated using HuRSTA-2a520709 Affymetrix arrays and normalized by the iterative rank order normalization method.

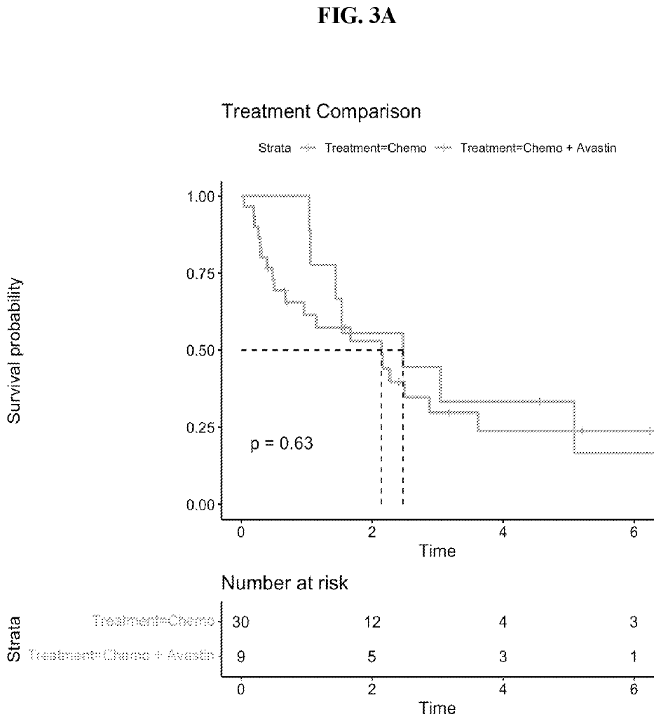

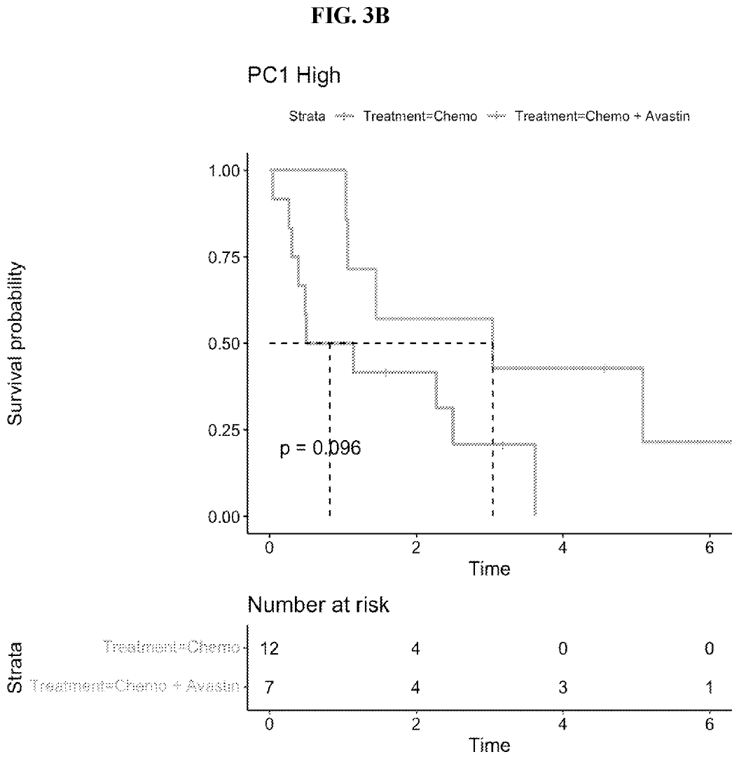

The cohort showed the AVASTIN®+Chemo group had a median OS of 2.47 years compared to 2.14 years in the Chemo group (p=0.63; FIG. 3A and Table 5). In contrast, patients classified as high PC1 group based on the 279 probe sets identified from the Moffitt training cohort had numerically improved median OS if AVASTIN®+Chemo was given (3.04 years in AVASTIN®+Chemo vs 0.82 year in Chemo; p=0.096; FIG. 3B and Table 6). Further evaluation showed a total of 18 probe sets with significant association with death risk (i.e., prognostic effect). Among them, there were 14 probe sets linked to 14 genes. The 14 significant genes in the Consortium cohort were BFSP2-AS1, ERICH1, HERC4, HIST2H2BF, IWS1, LINC01405, MCHR1, MTMR6, SLC30A2, SLITRK1, SRP68, SRRM4, TFCP2L1, ZNF462. Among them, there were 8 genes linked to the Avatar cohort (ERICH1, HERC4, HIST2H2BF, IWS1, MTMR6, SRP68, TFCP2L1, ZNF462). The Consortium significant genes showed robust predictive effect in the three cohort

TABLE 5
Median Survival Time of Treatment in Consortium Cohort

| strata | median | lower | upper |
|---|---|---|---|
| Treatment = Chemo | 2.139726 | 0.9589041 | NA |
| Treatment = Chemo + AVASTIN ® | 2.471233 | 1.4493151 | NA |

TABLE 6
Median Survival Time of Treatment in
High PC1 of the Consortium Cohort

| strata | median | lower | upper |
|---|---|---|---|
| Treatment = Chemo | 0.8205479 | 0.3945205 | NA |
| Treatment = Chemo + AVASTIN ® | 3.0410959 | 1.0602740 | NA |

### (5) Validation in Moffitt Avatar Cohort

Data were collected from 36 mCRC patients in the Moffitt Avatar cohort. Overall survival (OS) was the primary endpoint (n=36 death event; 61%). Twenty-five patients (69%) received Avastin and chemotherapy while 11 patients (31%) had chemotherapy only. Gene expression data was generated by RNA seq.

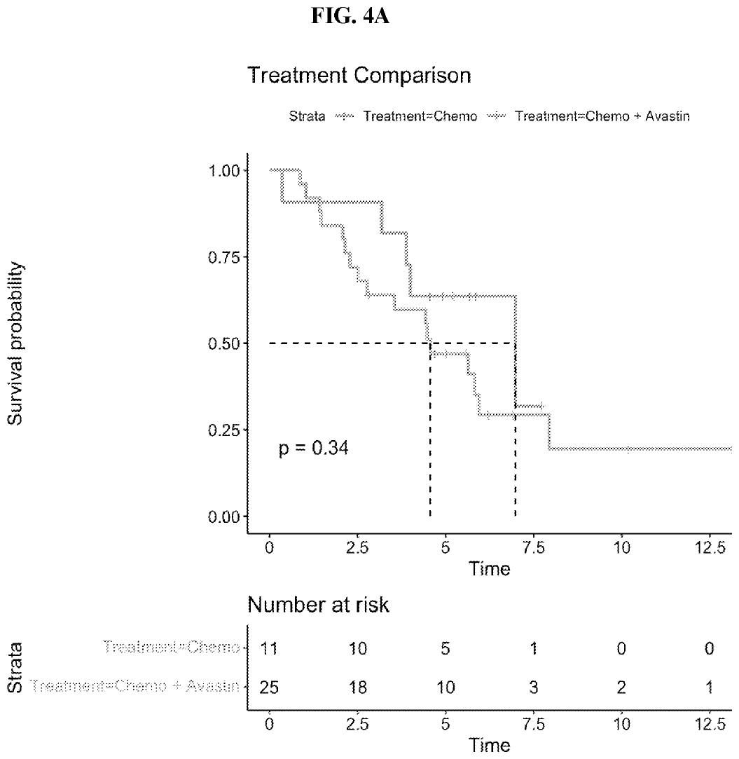

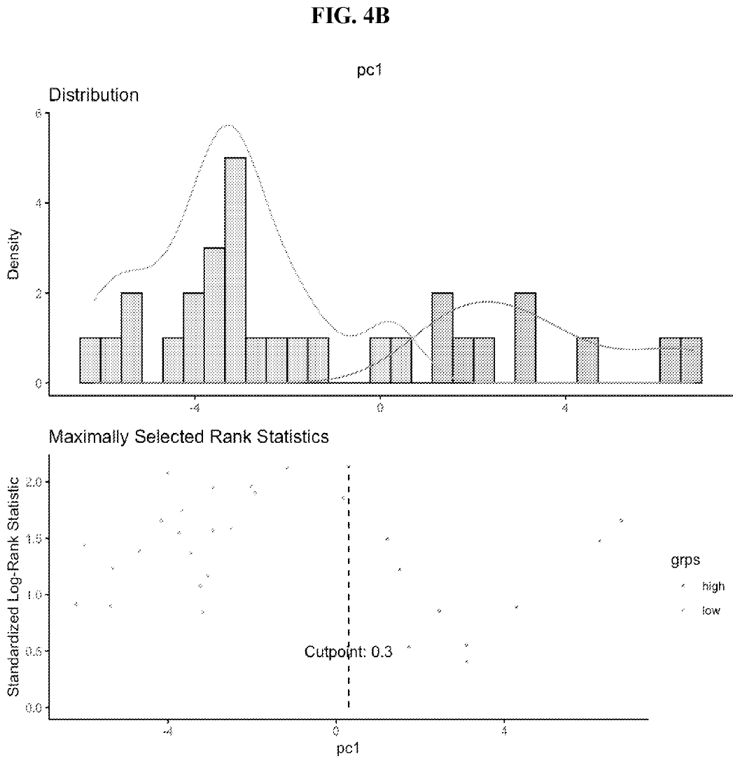

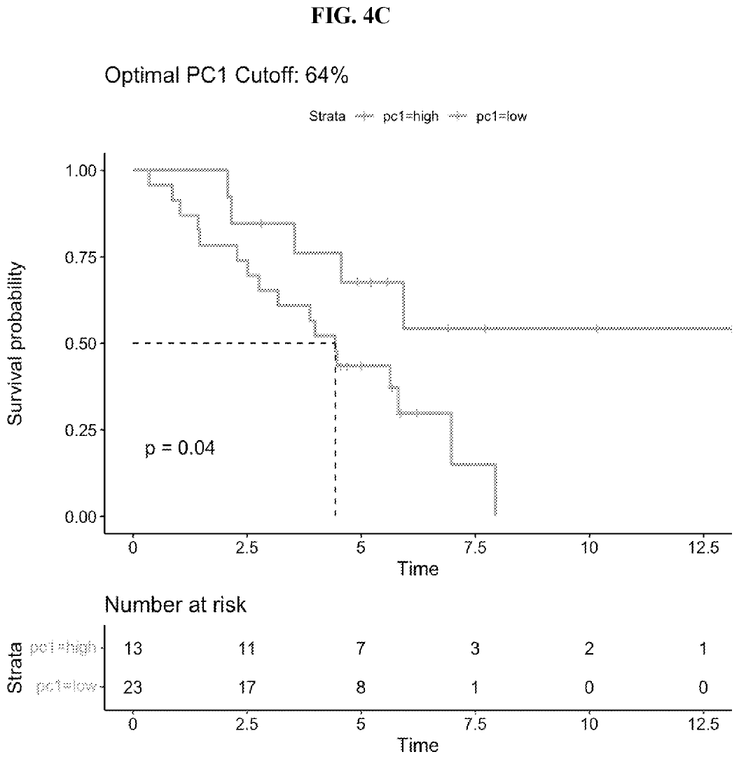

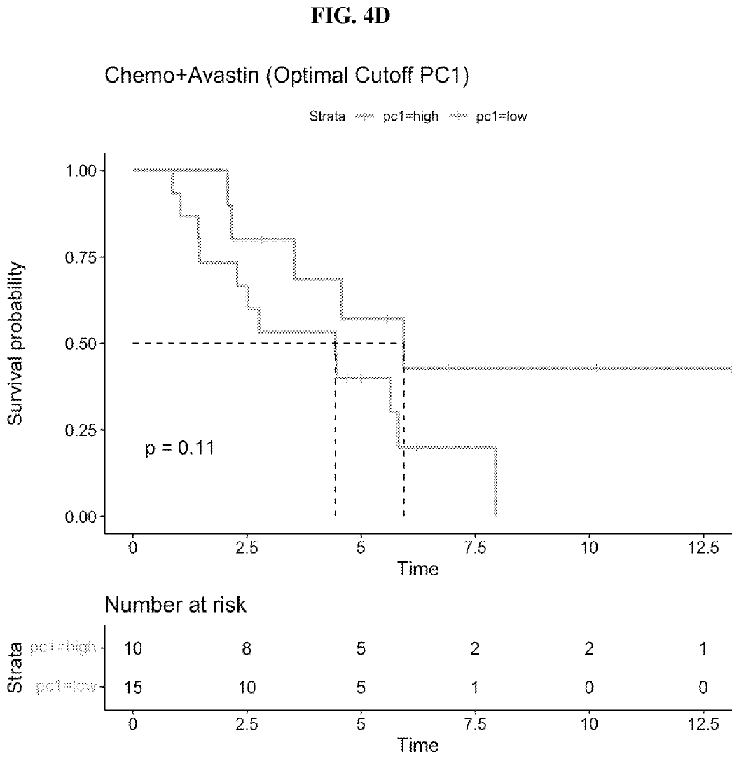

The cohort had a lower median OS (4.56 years) in the AVASTIN®+Chemo group versus 6.98 years in the Chemo group (p=0.34; FIG. 4A and Table 7). This pattern was different from the Moffitt training and Consortium cohorts with observed median OS higher in the AVASTIN®+Chemo group. Because of its distinctive characteristic, we focused on the overall group and the AVASTIN®+Chemo group to assess the signature effect. While the RNA-seq data used in this cohort had 59368 genes, 25% of them (14686 genes) had positive expression (>2) in at least 10% subjects. Furthermore, only 111 genes were able to link to the 279 probe sets in the Moffitt training cohort. Therefore, PC1 score was derived based on the 111 genes to validate the signature. The results showed an optimal PC1 cutoff at 0.3 (64%; FIG. 4B). The median OS was higher in high PC1 compared to low PC1 in the overall group (not reached vs 4.43 years; p=0.04; FIG. 4C and Table 8) and in the AVASTIN®+Chemo sub-group (5.94 vs 4.43 years; p=0.11; FIG. 4D and Table 9). Further evaluation showed 9 genes with significant negative association with death risk (i.e., low expression tended to increase death event). The 8 genes (CLPTM1, GTPBP1, IWS1, MAP3K14, MBD2, PIH1D1, SLC25A27, TJP2) after removing USP46 gene (maximum expression <10) showed robust predictive effect (prediction of AVASTIN®) in Moffitt training cohort. Also showed numerically improved OS for patients with high PC1 in overall and in the AVASTIN® group.

TABLE 7
Median Survival Time of Treatment in Avatar Cohort

| strata | median | lower | upper |
|---|---|---|---|
| Treatment = Chemo | 6.975343 | 3.991781 | NA |
| Treatment = Chemo + AVASTIN ® | 4.561644 | 2.764384 | NA |

$pc1

TABLE 8
Median Survival Time of PC1 Optimal
Cutoff (Overall) in Avatar Cohort

| strata | median | lower | upper |
|---|---|---|---|
| pc1 = high | NA | 4.561644 | NA |
| pc1 = low | 4.430137 | 2.764384 | NA |

TABLE 9
Median Survival Time of PC1 Optimal Cutoff
(AVASTIN ®) in Avatar Cohort

| strata | median | lower | upper |
|---|---|---|---|
| pc1 = high | 5.936986 | 3.539726 | NA |
| pc1 = low | 4.430137 | 2.279452 | NA |

### (6) Refinement of Avastin Gene Signature

(a) Signature Derived from Significant Genes in Consortium Cohort

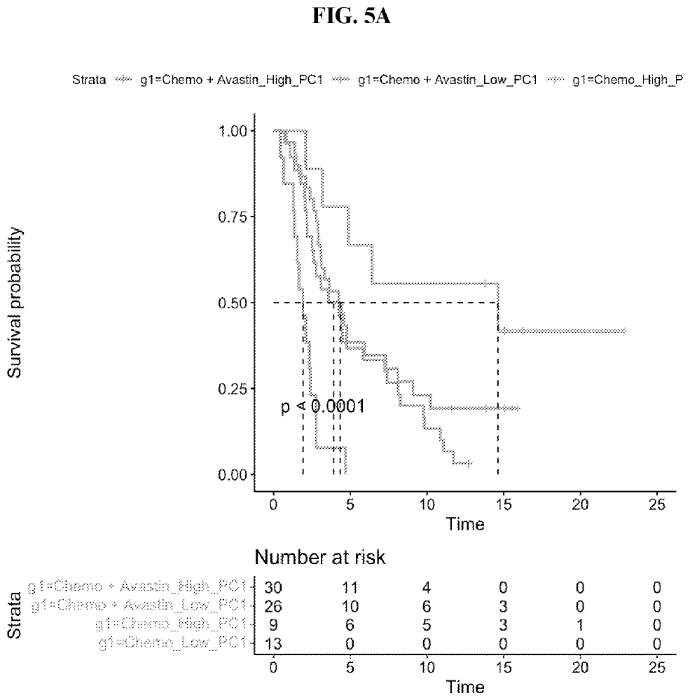

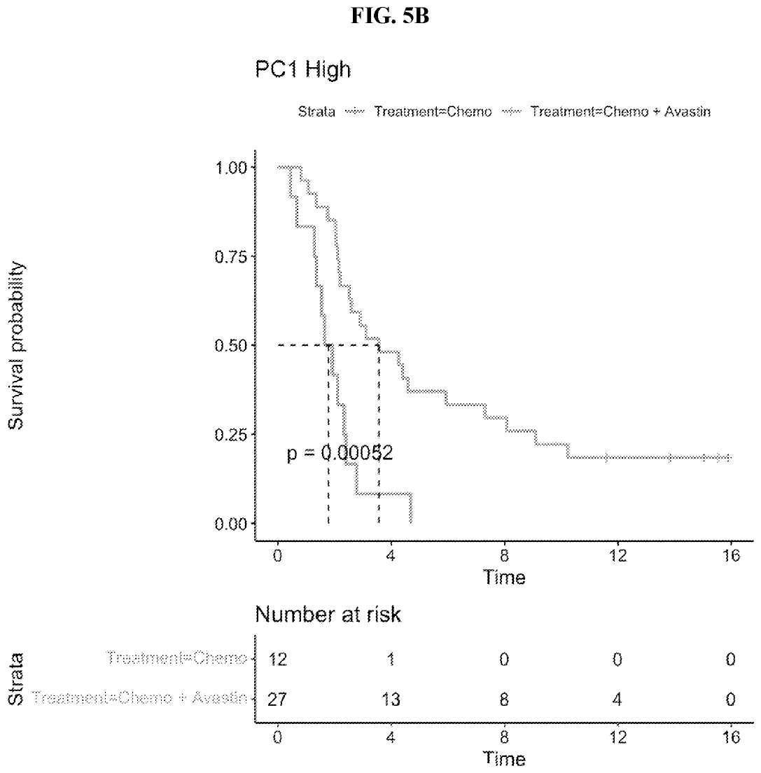

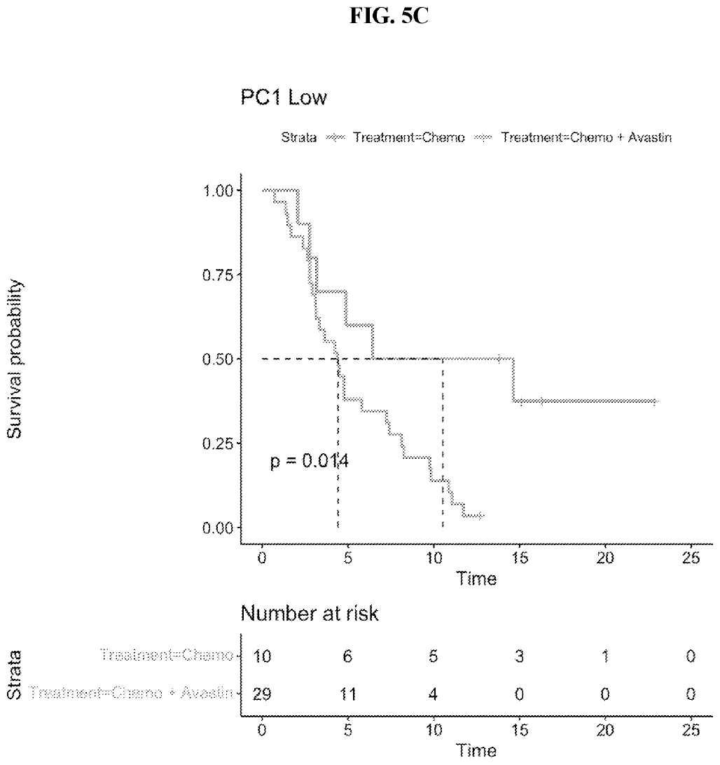

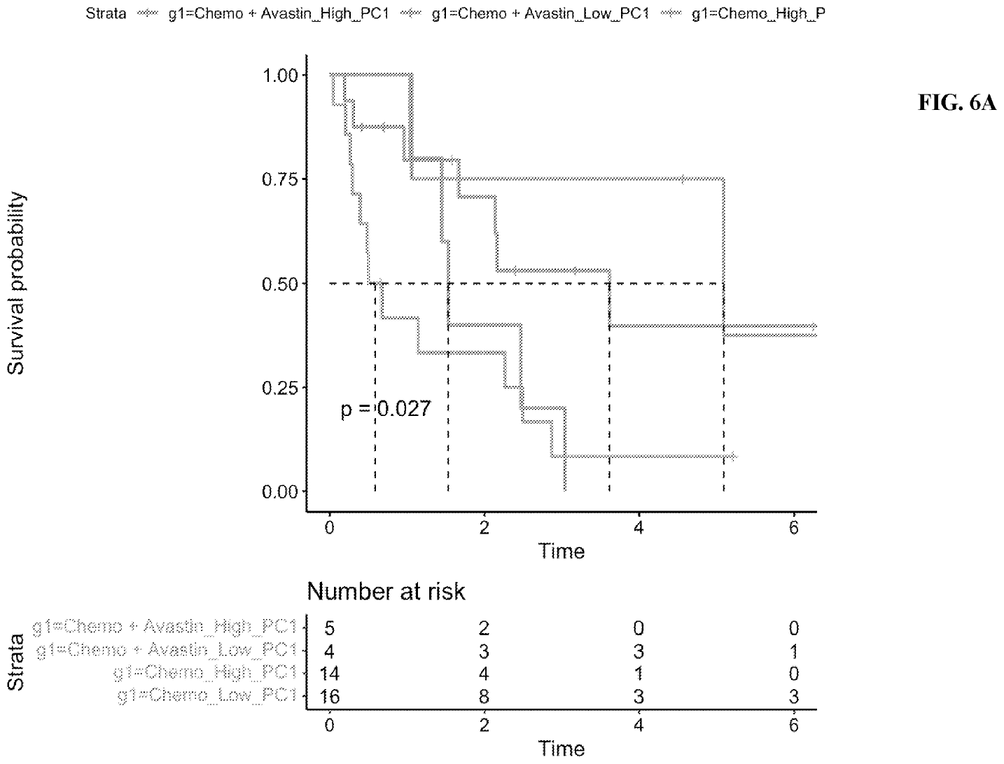

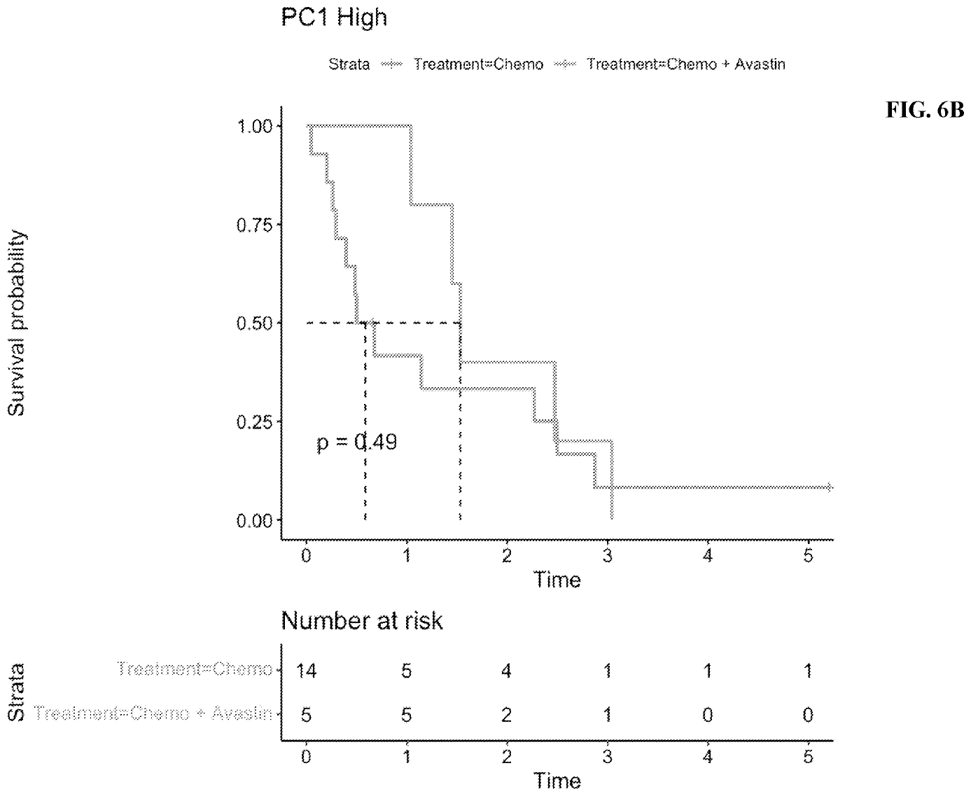

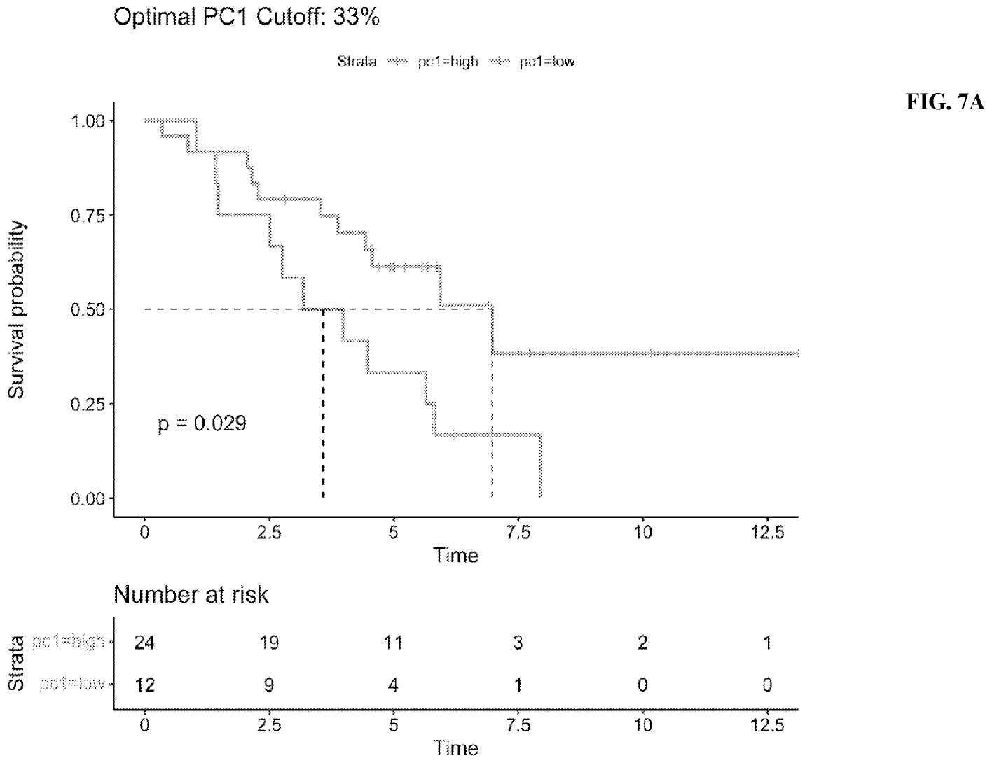

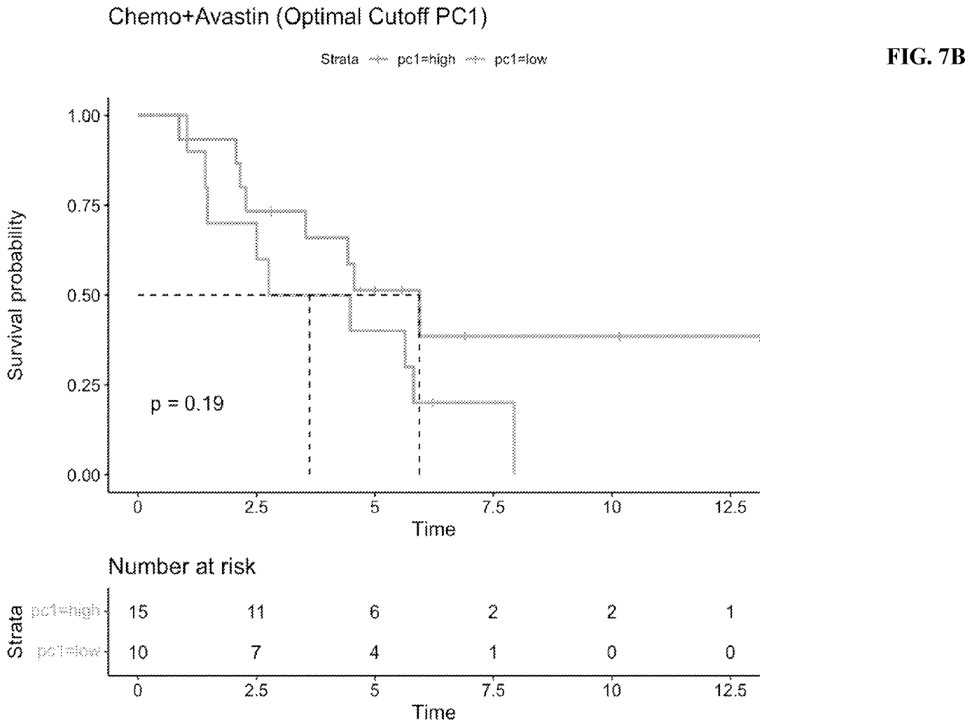

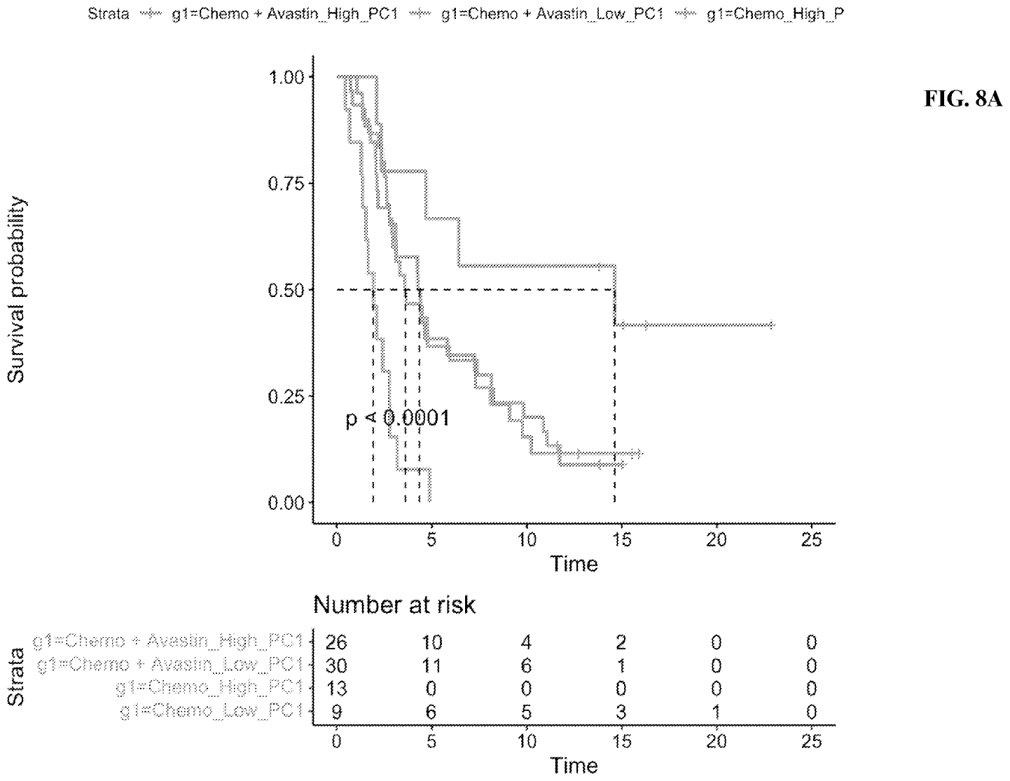

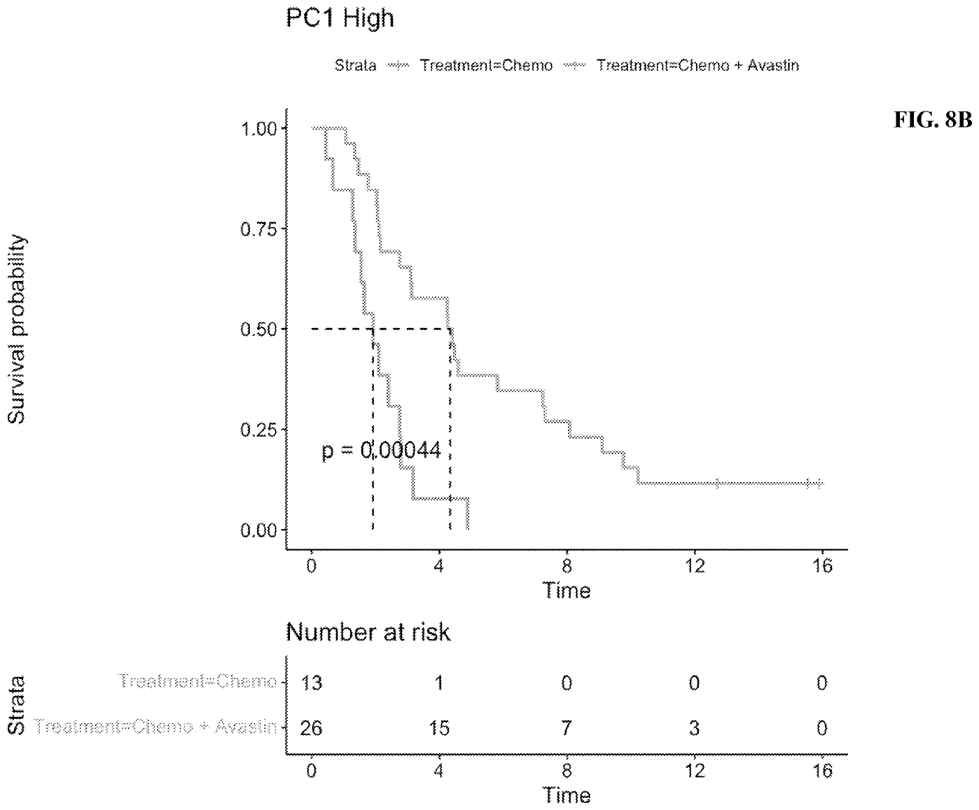

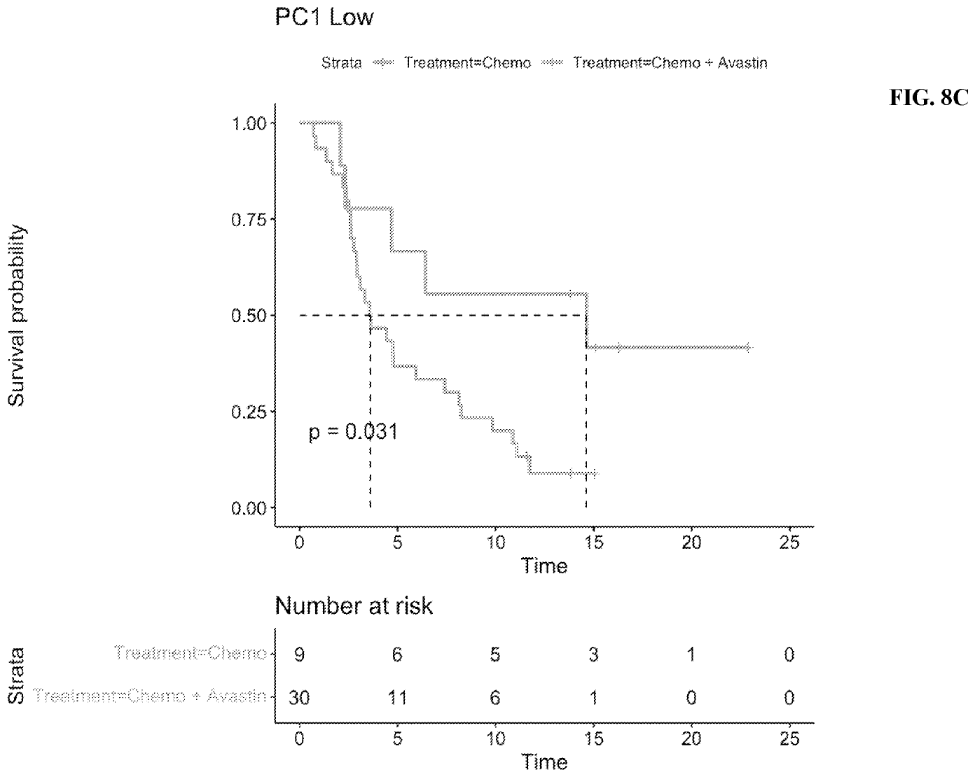

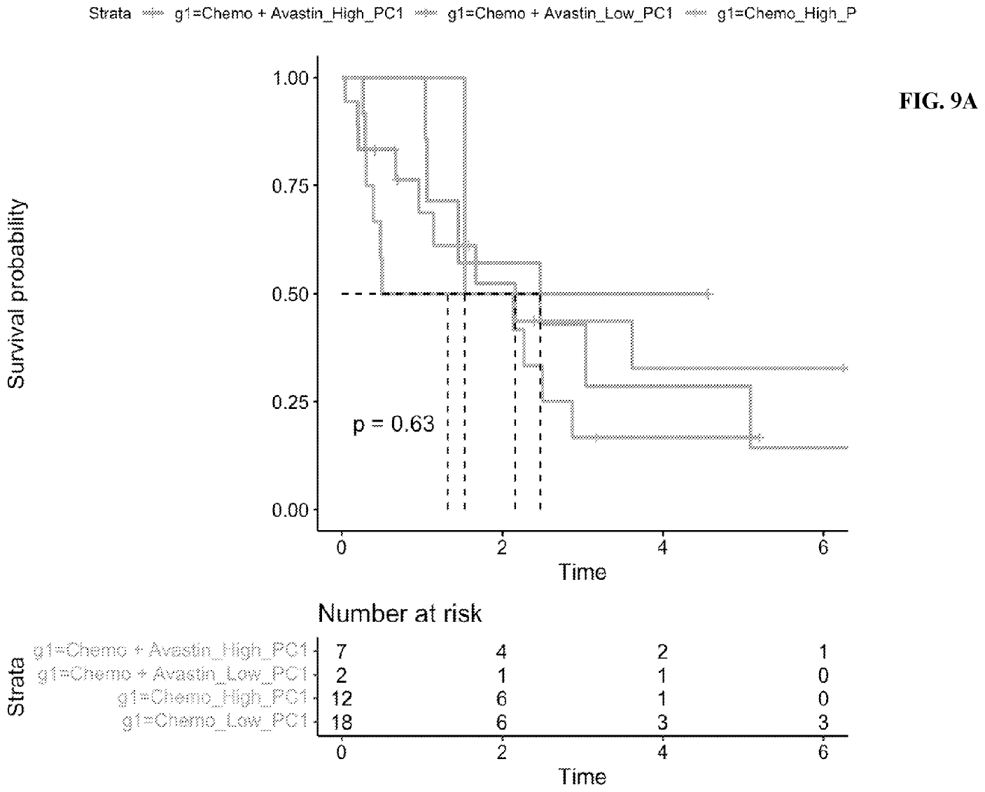

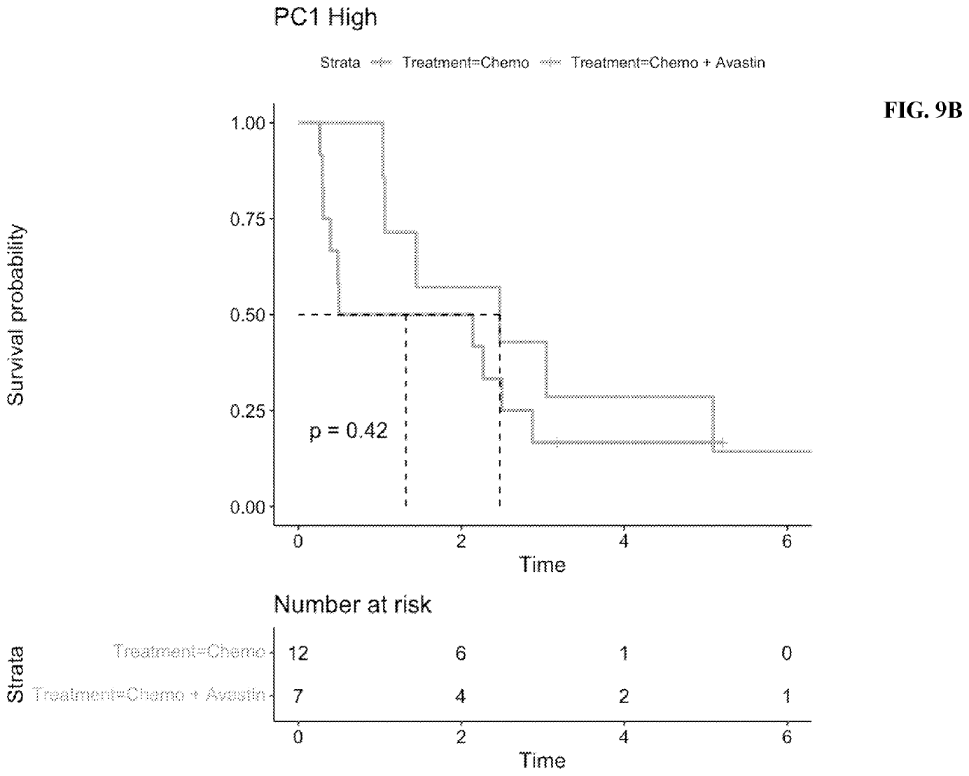

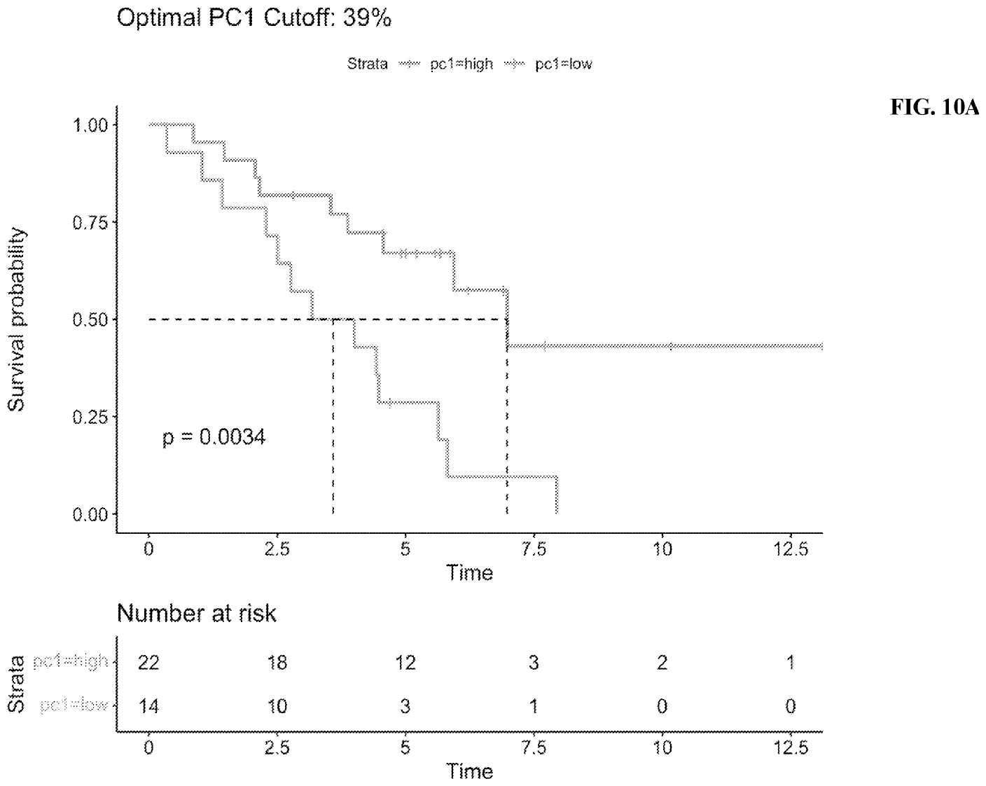

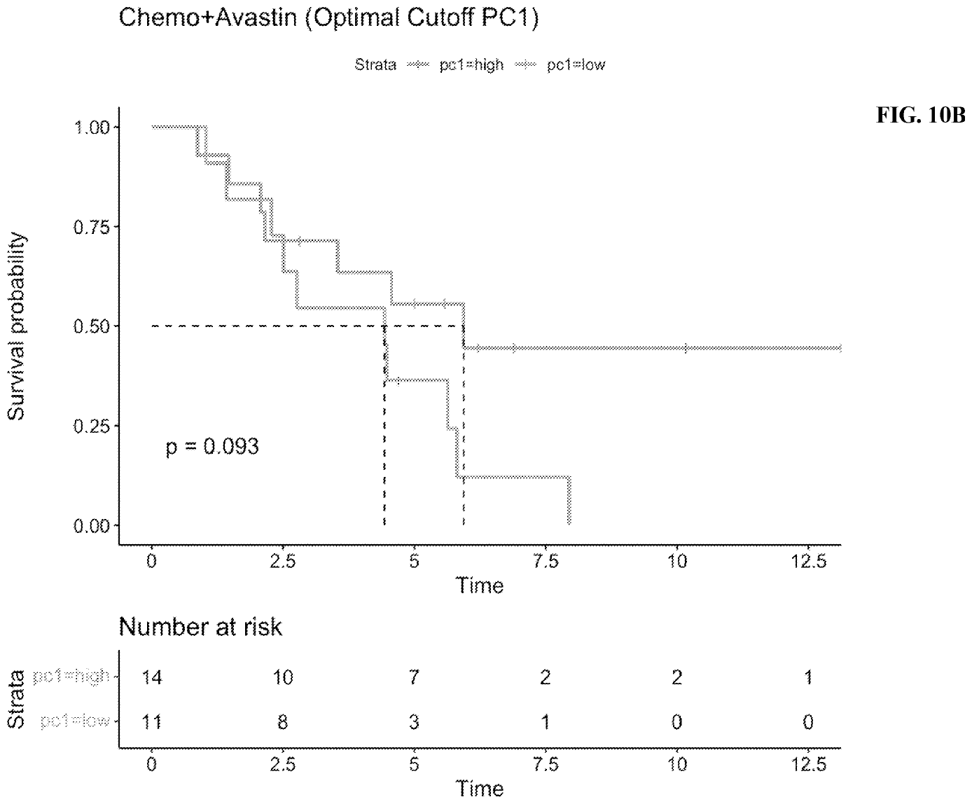

There were 7 significant genes in the Consortium cohort (HERC4, HIST2H2BF, IWS1, MCHR1, SLC30A2, SRP68, ZNF462) with the same up-regulated or down-regulated pattern. Among them, there were 5 genes linked to the Avatar cohort (HERC4, HIST2H2BF, IWS1, SRP68, ZNF462). The Consortium significant genes showed robust predictive effect in the three cohorts FIGS. 5A, 5B, and 5C).

TABLE 10
Median Survival Time of Interaction Model in Moffitt Cohort

| Strata | Median | Lower | upper |
|---|---|---|---|
| g1 = Chemo + Avastin_High_PC1 | 3.567123 | 2.509589 | 8.082192 |
| g1 = Chemo + Avastin_Low_PC1 | 4.419178 | 3.123288 | 7.391781 |
| g1 = Chemo_High_PC1 | 1.786301 | 1.356164 | NA |
| g1 = Chemo_Low_PC1 | 10.519178 | 3.178082 | NA |

Interaction Model: High PC1

TABLE 11
Median Survival Time of High PC1 in Moffitt Cohort

| Strata | Median | Lower | upper |
|---|---|---|---|
| Treatment = Chemo | 1.786301 | 1.356164 | NA |
| Treatment = Chemo + Avastin | 3.567123 | 2.509589 | 8.082192 |

Interaction Model: Low PC1

TABLE 12
Median Survival Time of Low PC1 in Moffitt Cohort

| Strata | Median | Lower | upper |
|---|---|---|---|
| Treatment = Chemo | 10.519178 | 3.178082 | NA |
| Treatment = Chemo + Avastin | 4.419178 | 3.123288 | 7.391781 |

Moffitt consortium cohort

TABLE 13
Median Survival Time of Interaction
Model in Moffitt Consortium Cohort

| Strata | Median | Lower | upper |
|---|---|---|---|
| g1 = Chemo + Avastin_High_PC1 | 1.5315068 | 1.4493151 | NA |
| g1 = Chemo + Avastin_Low_PC1 | 5.0876712 | 1.0602740 | NA |
| g1 = Chemo_High_PC1 | 0.5863014 | 0.3945205 | NA |
| g1 = Chemo_Low_PC1 | 3.6164384 | 1.6684932 | NA |

Interaction Model: High PC1

TABLE 14
Median Survival Time of High PC1 in Moffitt Consortium Cohort

| Strata | Median | Lower | upper |
|---|---|---|---|
| Treatment = Chemo | 0.5863014 | 0.3945205 | NA |
| Treatment = Chemo + Avastin | 1.5315068 | 1.4493151 | NA |

Moffitt Avatar Cohort

TABLE 15
Median Survival Time of PC1 in Moffitt Avatar Cohort

| Strata | Median | Lower | upper |
|---|---|---|---|
| PC1 = high | 6.975343 | 4.430137 | NA |
| PC1 = low | 3.584932 | 2.506849 | NA |

Avatar: Avastin subgroup

TABLE 16
Median Survival Time of Avastin in Moffitt Avatar Cohort

| Strata | Median | Lower | upper |
|---|---|---|---|
| PC1 = high | 6.975343 | 4.430137 | NA |
| PC1 = low | 3.584932 | 2.506849 | NA |

Signature derived from significant genes in Avatar cohort

The 8 genes (CLPTM1, GTPBP1, IWS1, MAP3K14, MBD2, PIH1D1, SLC25A27, TJP2) after removing USP46 gene (maximum expression <10) showed robust predictive effect (prediction of Avastin) in Moffitt training cohort and also showed numerically improved OS for patients with high PC1 in overall and in the Avastin group.

TABLE 17
Median Survival Time of Interaction Model in Moffitt Cohort

| Strata | Median | Lower | upper |
|---|---|---|---|
| g1 = Chemo + Avastin_High_PC1 | 4.336986 | 2.761644 | 8.082192 |
| g1 = Chemo + Avastin_Low_PC1 | 3.601370 | 2.898630 | 8.126027 |
| g1 = Chemo_High_PC1 | 1.923288 | 1.356164 | NA |
| g1 = Chemo_Low_PC1 | 14.621918 | 4.687671 | NA |

Interaction Model: High PC1

TABLE 18
Median Survival Time of High PC1 in Moffitt Cohort

| Strata | Median | Lower | upper |
|---|---|---|---|
| Treatment = Chemo | 1.923288 | 1.356164 | NA |
| Treatment = Chemo + Avastin | 4.336986 | 2.761644 | 8.082192 |

Interaction Model: Low PC1

TABLE 19
Median Survival Time of Low PC1 in Moffitt Cohort

| Strata | Median | Lower | upper |
|---|---|---|---|
| Treatment = Chemo | 14.621918 | 4.687671 | NA |
| Treatment = Chemo + Avastin | 3.601370 | 2.898630 | 8.126027 |

Moffitt Consortium cohort

TABLE 20
Median Survival Time of Interaction
Model in Moffitt Consortium Cohort

| Strata | Median | Lower | upper |
|---|---|---|---|
| g1 = Chemo + Avastin_High_PC1 | 2.471233 | 1.0602740 | NA |
| g1 = Chemo + Avastin_Low_PC1 | 1.531507 | 1.5315068 | NA |
| g1 = Chemo_High_PC1 | 1.319178 | 0.3945205 | NA |
| g1 = Chemo_Low_PC1 | 2.161644 | 0.9589041 | NA |

Interaction Model: High PC1

TABLE 21
Median Survival Time of High PC1 in Moffitt Consortium Cohort

| Strata | Median | Lower | upper |
|---|---|---|---|
| Treatment = Chemo | 1.319178 | 0.3945205 | NA |
| Treatment = Chemo + Avastin | 2.471233 | 1.0602740 | NA |

Moffitt Avatar cohort

TABLE 22
Median Survival Time of PC1 in Moffitt Avatar Cohort

| Strata | Median | Lower | upper |
|---|---|---|---|
| PC1 = high | 6.975343 | 4.561644 | NA |
| PC1 = low | 3.584932 | 2.506849 | NA |

Avatar: Avastin subgroup

TABLE 23
Median Survival Time of Avastin in Moffitt Avatar Cohort

| Strata | Median | Lower | upper |
|---|---|---|---|
| PC1 = high | 5.936986 | 3.539726 | NA |
| PC1 = low | 4.430137 | 2.279452 | NA |

1. A kit used for treating cancer comprising one or more probe sets comprising merck2_AA101946_x_at, merck2_AA490328_at, merck2_AF129457_at, merck2_AF289590_at, merck2_AI091276_at, merck2_AI223270_at, merck2_AI962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at, merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_O32_up_at, merck2_VZV_OKA_O47_up_at, merck2_XM_938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655_s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at, merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_BI045425_at, merck_BI757398_at, merck_BM676784_at, merck_BM805663_at, merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431_s_at, merck_NM_001494_at, merck_NM_001883_at, merck_NM_001993_at, merck_NM_002501_s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41_s_at, merck_VZV_OKA_ORF9_s_at, merck_X85629_at, merck_X98206_at, merck_XM_063314_at, merck_XM_210303_at, merck_XM_373233_x_at, merck_XM_378090_at, merck_XM_926011_at, merck_XM_928173_s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, or merck_hCT1846664_1_at, merck_hCT2307348_at, or merck_hsa_mir_153_1_x_at.

2. The kit of claim 1, wherein the one or more probe sets bind to one or more genes comprising TIMM8A, PRDM15, LOC100130744, LANCL2, LOC101929529, NPHP3 NPHP3-ACAD11, MYO5B, ZSCAN30, ZNF462, CDON, ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1.

3. The kit of claim 1, wherein the cancer to be treated comprises colorectal cancer.

4. The kit of claim 3, wherein the colorectal cancer to be treated comprises metastatic colorectal cancer.

5. (canceled)

6. A method of treating a cancer in a subject comprising:
a) obtaining a cancerous tissue sample from the subject;
b) contacting the cancerous tissue sample with one or more probe sets that bind to one or more genes that are differentially expressed in the cancer;
c) assaying the expression of one or more genes from the cancerous tissue sample to create a gene signature, wherein principle component analysis is applied to the gene signature to create a probability score for the subject; and
d) treating the subject with chemotherapy and bevacizumab when the probability score is high; and treating the subject with chemotherapy and not bevacizumab when the probability score is low;
wherein the one or more probe sets comprise merck2_AA101946_x_at, merck2_AA490328_at, merck2_AF129457_at, merck2_AF289590_at, merck2_AI091276_at, merck2_AI223270_at, merck2_AI962276_at, merck2_AK092910_at, merck2_AK093149_at, merck2_AK131291_at, merck2_AL359561_at, merck2_AL832223_at, merck2_AL834299_at, merck2_AL834442_at, merck2_AM392650_at, merck2_AW007481_at, merck2_AX721105_at, merck2_BC001038_at, merck2_BC033091_at, merck2_BC035840_at, merck2_BC039861_at, merck2_BE249972_x_at, merck2_BE349054_x_at, merck2_BF248252_at, merck2_BF591243_at, merck2_BF692777_at, merck2_BG896934_at, merck2_BI047264_at, merck2_BM545067_s_at, merck2_BM681401_at, merck2_BU618242_at, merck2_BX115227_at, merck2_BX647415_at, merck2_BX647735_at, merck2_BX648311_at, merck2_BY799718_at, merck2_CMV_UL43_at, merck2_CR749241_s_at, merck2_CX783886_a_at, merck2_DB492896_at, merck2_DQ891843_at, merck2_ENST00000355104_x_at, merck2_ENST00000371290_s_at, merck2_F25718_at, merck2_HSV1_ORF_UL46_at, merck2_HSV2_ORF_UL18_at, merck2_JC_vir_smTAg_at, merck2_NM_001005224_s_at, merck2_NM_001037866_s_at, merck2_NM_001080537_at, merck2_NM_005570_at, merck2_NM_006485_at, merck2_NM_006594_at, merck2_NM_022079_at, merck2_NM_022747_at, merck2_NM_025214_at, merck2_NM_030813_at, merck2_NM_032260_s_at, merck2_NM_032639_at, merck2_NM_052996_a_at, merck2_NM_205833_at, merck2_VZV_OKA_032_up_at, merck2_VZV_OKA_047_up_at, merck2_XM 938193_at, merck_AB058756_a_at, merck_AF038451_a_at, merck_AF085981_at, merck_AF118083_a_at, merck_AF143326_at, merck_AF147412_at, merck_AF147431_at, merck_AF161340_at, merck_AF169158_a_at, merck_AF311308_at, merck_AI204893_at, merck_AJ406951_s_at, merck_AJ459838_at, merck_AK000932_at, merck_AK021682_at, merck_AK022168_at, merck_AK022347_at, merck_AK022443_at, merck_AK023601_at, merck_AK024198_at, merck_AK024231_at, merck_AK024810_a_at, merck_AK025202_a_at, merck_AK025621_at, merck_AK026808_at, merck_AK054931_at, merck_AK055172_a_at, merck_AK055584_at, merck_AK091999_at, merck_AK093735_s_at, merck_AK095449_at, merck_AK096712_at, merck_AK097571_s_at, merck_AK097840_a_at, merck_AK123535_at, merck_AK123655_s_at, merck_AK123888_at, merck_AK125149_at, merck_AK127206_s_at, merck_AK130508_at, merck_AK222828_a_at, merck_AL044815_at, merck_AL133569_at, merck_AL162044_at, merck_AL353936_at, merck_AL563761_at, merck_AV762307_at, merck_AW370282_at, merck_AW665698_at, merck_AW771625_a_at, merck_AW809224_at, merck_AW843987_at, merck_AW977003_at, merck_AY054391_at, merck_AY147849_a_at, merck_BC001905_a_at, merck_BC007984_at, merck_BC012447_a_at, merck_BC014119_at, merck_BC015877_a_at, merck_BC019893_s_at, merck_BC022892_at, merck_BC034596_at, merck_BC036594_at, merck_BC039321_at, merck_BC064385_at, merck_BC110326_at, merck_BC110867_s_at, merck_BE219852_at, merck_BE463518_at, merck_BE546828_at, merck_BF342524_at, merck_BF679703_at, merck_BF965720_at, merck_BG236239_a_at, merck_BG287007_at, merck_BG675291_at, merck_BG740109_a_at, merck_BG943385_at, merck_BG956704_x_at, merck_BI045425_at, merck_BI757398_at, merck_BM676784_at, merck_BM805663_at, merck_BQ380337_at, merck_BU145850_at, merck_BU935068_a_at, merck_BX095858_at, merck_BX096093_at, merck_BX097755_a_at, merck_BX102791_at, merck_BX102980_at, merck_BX116278_at, merck_BX119057_at, merck_BX329942_at, merck_CA309176_at, merck_CA942562_at, merck_CD102009_at, merck_CN388529_a_at, merck_CR742243_at, merck_CR994266_a_at, merck_CV392556_at, merck_CX785712_at, merck_DA214150_a_at, merck_DA234289_at, merck_DB351522_at, merck_DB546490_at, merck_DN831737_at, merck_DV080080_at, merck_ENST00000248668_at, merck_ENST00000316506_a_at, merck_ENST00000323496_x_at, merck_ENST00000327196_at, merck_ENST00000331146_at, merck_ENST00000341244_a_at, merck_ENST00000359421_at, merck_ENST00000366485_at, merck_ENST00000376325_at, merck_ENST00000378999_s_at, merck_ENST00000381435_at, merck_G65625_at, merck_NC_001405_ORF_1045_s_at, merck_NM_000258_at, merck_NM_000273_at, merck_NM_000782_at, merck_NM_000873_at, merck_NM_001001706_at, merck_NM_001002247_at, merck_NM_001004434_s_at, merck_NM_001004752_at, merck_NM_001012505_a_at, merck_NM_001024599_s_at, merck_NM_001039753_s_at, merck_NM_001039_at, merck_NM_001294_at, merck_NM_001431_s_at, merck_NM_001494_at, merck_NM_001883_at, merck_NM_001993_at, merck_NM_002501_s_at, merck_NM_003920_s_at, merck_NM_003954_at, merck_NM_004318_at, merck_NM_004387_at, merck_NM_004422_at, merck_NM_004683_s_at, merck_NM_005184_s_at, merck_NM_006143_at, merck_NM_006268_at, merck_NM_007179_at, merck_NM_014184_s_at, merck_NM_014230_s_at, merck_NM_014553_at, merck_NM_015058_at, merck_NM_015832_a_at, merck_NM_016593_at, merck_NM_017916_at, merck_NM_018055_at, merck_NM_018219_at, merck_NM_020457_at, merck_NM_020651_at, merck_NM_020677_s_at, merck_NM_020774_at, merck_NM_021912_at, merck_NM_021946_s_at, merck_NM_021983_x_at, merck_NM_022358_at, merck_NM_025214_at, merck_NM_030944_at, merck_NM_032143_a_at, merck_NM_032594_at, merck_NM_052910_at, merck_NM_058165_at, merck_NM_058219_at, merck_NM_133262_at, merck_NM_133492_at, merck_NM_138447_a_at, merck_NM_139174_at, merck_NM_139315_s_at, merck_NM_145270_at, merck_NM_152730_s_at, merck_NM_153274_at, merck_NM_173798_at, merck_NM_174962_x_at, merck_NM_176889_at, merck_NM_178012_at, merck_NM_181608_x_at, merck_NM_182505_at, merck_NM_182800_at, merck_NM_182905_s_at, merck_NM_198994_at, merck_NM_199180_at, merck_VZV_OKA_ORF41_s_at, merck_VZV_OKA_ORF9_s_at, merck_X85629_at, merck_X98206_at, merck_XM_063314_at, merck_XM_210303_at, merck_XM_373233_x_at, merck_XM_378090_at, merck_XM_926011_at, merck_XM_928173_s_at, merck_XM_928586_x_at, merck_XM_929767_at, merck_XM_933463_at, merck_XM_937104_at, merck_XM_940419_at, merck_XM_943662_at, merck_XM_945363_at, merck_hCT1651346_at, merck_hCT1651838_3_at, merck_hCT1812405_2_at, merck_hCT1846664_1_at, or merck_hCT2307348_at, or merck_hsa_mir_153_1_x_at.

7. (canceled)

8. The method of claim 6, wherein the one or more genes comprise TIMM8A, PRDM15, LOC100130744, LANCL2, LOC101929529, NPHP3 NPHP3-ACAD11, MYO5B, ZSCAN30, ZNF462, CDON, ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1.

9. The method of claim 6, wherein the subject has a low probability score and is already receiving chemotherapy, and wherein bevacizumab is not administered.

10. The method of claim 6, wherein the subject has a high probability score and is already receiving chemotherapy, and wherein bevacizumab is not administered.

11. The method of claim 6, wherein the cancer comprises colorectal cancer.

12. The method of claim 11, wherein the colorectal cancer comprises metastatic colorectal cancer.

13. A method of treating a cancer in a subject comprising
a) obtaining a cancerous tissue sample from the subject;
b) contacting the cancerous tissue sample with one or more probe sets that bind to one or more genes that are differentially expressed in the cancer;
c) assaying the expression of one or more genes from the sample to create a gene signature, wherein principle component analysis is applied to the gene signature to create a probability score for the subject; and
d) treating the subject with chemotherapy and bevacizumab when the probability score is high; and treating the subject with chemotherapy and not bevacizumab when the probability score is low;
wherein the one or more genes comprise TIMM8A, PRDM15, LOC100130744, LANCL2, LOC101929529, NPHP3 NPHP3-ACAD11, MYO5B, ZSCAN30, ZNF462, CDON, ZNF689, NCOA7, ZNF280D, NFIX, ZNF467, EPN3, SLC25A27, GJC2, NODAL, RPL17 RPL17-C18orf32, TDRKH, FAM149B1, PPIE, FIP1L1, MRPL38, GABPB1-AS1, GALNT1, ERICH1, CEP126, AP3S1, AHI1, MRPL1, FRMPD2, UBE3A, ZNF365, ICAM2, PRR21, CT47A1 CT47A10 CT47A11 CT47A12 CT47A2 CT47A3 CT47A4 CT47A5 CT47A6 CT47A7 CT47A8 CT47A9, LINC01405, OR4F16 OR4F21 OR4F29 OR4F3, RGPD5 RGPD6, SNTN, LMAN1, FBLN1m AP4B1, HERC4, CCDC85C, CCDC68, CLPB, RGPD5 RGPD6, PLEKHA8, PRDM7, IGSF1, SH2D1A STAG2 HSPA8P20 NPM1P34 TEX13D ZIK1P1 LOC101928402, SRRM4, AGR2, ENTPD4, EXOC5, FOXP1 FOXP1-AS1 MIR1284, ARID1B MIR4466, DIS3L, CABP5, MGAT1, KRTAP9-9, OR2H1, COL9A2, CSTF2T MIR605 PRKG1 RSU1P3 LOC102724719, CSTF2T MIR605 PRKG1 RSU1P3, LOC102724719, FOXP1 FOXP1-AS1 MIR1284, EFR3A, PRPF40A, LARGE1, LOC101927365, HERC4, MIR9-3HG, DLG5, STT3B, LOC101929713, CEP83, CRK, USP46, LOC105378730, LOC105379589 LOC107984097 LOC107987409 LOC107987410, FBXL18, UCHL5, ZNF555, CD99P1, LONRF2, LOC100507281, LMAN2L, MTMR6, LOC107987180, PCDH9 PCDH9-AS4, PSD3, USP3-AS1, MAP3K6, CSNK1G1, GTPBP1, BDNF-AS, MIB1, PCSK2, BFSP2-AS1, SP110, LINC00824, CDH19, CD99P1, IWS1, LINC01725, TISP43 LOC646743, TRIQK, CDH24, LOC101927472, LINC02004, SIRPD, CLYBL, THUMPD3-AS1, KLRA1P, LINC01971, SOGA1, RIMBP2, TJP2, NFATC3 RPS12P27, GDAP2, MSN, ELFN1-AS1, PNPO, LOC101929371, LAMB1, JAK1, NDFIP1, UHRF2, RIN2, SLC25A48, PHF2P2, LRFN1, OR52B1P, BCAS4, LOC107985946, RPS17P5, TAF4B, FAM92A, OR14A2, SHISA7, REXO1L2P, MCHR1, MYL3, GPR143, CYP24A1, ICAM2, AK9, ANAPC11, SLC30A2, OR51F1, FOXP1, HIST2H2BF, EML6, SCNN1G, CLPTM1, EPB41L2, GDI2, CRHR2, F3, NFIX, TIMELESS, MAP3K14, ASPH, NKX2-5, DVL2, RGN, CALM3, GPR19, DPF2, INSL6, CNIH4, SRP68, TFCP2L1, VWA8, MBD2, CYP39A1, PIH1D1, NODAL, CCDC87, THAP11, PELI1, NMRAL1, MIB1, GABRB3, BCORL1, HLA-DRB4, KCNK15, CCDC68, LINC00597, ZRANB3, INSM2, SLITRK1, MOGAT1, EXOSC6, ATP6V1G3, ACER1, ZNF689, ADAD2, TAF6, PRR35, TBC1D32, BEST4, ZCCHC12, SSX9, TAS2R20, TUBB2B, KRTAP19-2, C9orf85, SRRT, WASHC1, TGM6, KIRREL2, ARID1B MIR4466, OR11H5P, LOC642515, PPIAL4A PPIAL4C PPIAL4D PPIAL4E PPIAL4F PPIAL4G, FAM207BP FAM207CP, SLC9B1P1, RPS28P1, LOC100288437, ELOCP31, or MIR153-1.

14. The method of claim 13, wherein the subject has a low probability score and is already receiving chemotherapy, and wherein bevacizumab is not administered.

15. The method of claim 13, wherein the subject has a high probability score and is already receiving chemotherapy, and wherein bevacizumab is not administered.

16. The method of treating a cancer of claim 13, wherein the cancer comprises colorectal cancer.

17. The method of treating a cancer of claim 16, wherein the colorectal cancer comprises metastatic colorectal cancer.
